(19) United States
(12) Patent Application Publication
(10) Pub. No.: US 2009/0111735 A1
(43) Pub. Date: Apr. 30, 2009

(54) Compounds and methods for reducing undesired toxicity of chemotherapeutic agents

(75) Inventors: Frederick H. Hausheer, Boerne, TX (US); Hary Kochat, San Antonio, TX (US); Qiuli Huang, San Antonio, TX (US)

Correspondence Address: Scott A. Whitaker, Senior Intellectual Property;Counsel, BioNumerik Pharmaceuticals, Inc., 8122 Datapoint Drive, Suite 1250, San Antonio, TX, 78229, US

(73) Assignee: BioNumerik Pharmaceuticals, Inc.

(21) Appl. No.: 12/283,995

(22) Filed: Sep. 17, 2008

Related U.S. Application Data
(63) Continuation-in-part of application No. 10/843,930, filed on May 12, 2004, now abandoned.

(51) Int. Cl. A61K 38/10; A61K 38/06; A61K 38/07; A61P 43/00; A61K 38/05; A61K 38/08

(52) U.S. Cl. 514/7; 514/18; 514/17; 514/16; 514/15; 514/19

(57) ABSTRACT

Novel compositions and formulations are disclosed that have use to mitigate or prevent physiologically deleterious side-effects and/or modulate the intracellular balance of oxidized and reduced thioredoxin (Trx) in patients with cancer who are receiving treatment with one or more chemotherapeutic agents. The compositions of matter are amino acid and peptide heteroconjugated disulfides of 2-mercaptoethane sulfonate sodium (mesna). In addition, methods of administration and kits comprising these novel mesna heteroconjugates are disclosed.

### RELATED APPLICATIONS

The present patent application is a Continuation-in-Part of U.S. patent application Ser. No. 10/843,930, filed on May 12, 2004 and entitled “COMPOUNDS AND METHODS FOR REDUCING UNDESIRED TOXICITY OF CHEMOTHERAPEUTIC AGENTS”, which is incorporated herein by reference in its entirety.

### FIELD OF THE INVENTION

The present invention relates to novel compositions of matter, namely certain short-chain peptides, and short chain peptides conjugated with a thioalkane sulfonate or phosphonate salt. The present invention also relates to pharmaceutical formulations and methods of administration for these novel formulations, which when administered to patients also receiving chemotherapy for cancer or other diseases, are useful as protective agents to mitigate or prevent the undesired toxic effects of the chemotherapeutic agent(s) and/or to modulate the intracellular balance of oxidized and reduced thioredoxin (Trx).

### BACKGROUND OF THE INVENTION

### I. Oxidative Metabolism

In its most simple terms, oxidative metabolism refers to the enzymatic pathways leading to the addition of oxygen (i.e., oxidation) or the removal of electrons or hydrogen (i.e., reduction) from intermediates in the pathways. The redox state of any particular biological environment can be defined as the sum of oxidative and reductive processes occurring within that environment which, in turn, directly relates to the extent to which molecules are oxidized or reduced within it. The redox potential of biological ions or molecules is a measure of their tendency to lose an electron (i.e., thereby becoming oxidized) and is expressed as E₀ in volts. The more strongly reducing an ion or molecule, the more negative its E₀. As previously stated, under normal physiological circumstances, most intracellular biological systems are predominantly found in a reduced state. Within cells, thiols (R—SH) such as glutathione (GSH), cysteine, homocysteine, and the like, are maintained in their reduced state, as are the nicotinamide nucleotide coenzymes NADH and NADPH. The opposite relationship is found in plasma, where the high partial pressure of oxygen (pO₂) promotes an oxidative environment, thereby leading to a high proportion (i.e., greater than 90%) of the physiological sulfur-containing amino acids and peptides (e.g., glutathione (GSH)) existing in stable oxidized (disulfide) forms. In plasma, there are currently no known enzymes that appear to reduce the disulfide forms of these sulfur-containing amino acids and GSH; this further contributes to the plasma vs. cellular disparity in terms of the relative proportions of physiological disulfides vs. thiols. Physiological circumstances can, however, arise which alter the overall redox balance and lead to a more oxidizing environment in the cell. Various complex physiological systems have evolved to remove, repair, and control the normal reducing environment. However, when the oxidizing environment overwhelms these protective mechanisms, oxidative damage and profound biological and toxic activity can occur.

In biological systems, the formation of potentially physiologically-deleterious reactive oxygen species (ROS) and that of reactive nitrogen species (RNS), may be caused from a variety of metabolic and/or environmental processes. By way of non-limiting example, intracellular ROS (e.g., hydrogen peroxide: H₂O₂; superoxide anion: O₂⁻; hydroxyl radical: OH⁻; nitric oxide: NO; and the like) may be generated by several mechanisms: (i) by the activity of radiation, both exciting (e.g., UV-rays) and ionizing (e.g., X-rays); (ii) during xenobiotic and drug metabolism; and (iii) under relative hypoxic, ischemic and catabolic metabolic conditions, as well as by exposure to hyperbaric oxygen. Protection against the harmful physiological activity of ROS and RNS species is mediated by a complex network of overlapping mechanisms and metabolic pathways that utilize a combination of small redox-active molecules and enzymes coupled with the expenditure of reducing equivalents. These complex networks of mechanisms, metabolic pathways, small redox-active molecules, and enzymes will be fully discussed, infra.

Concentrations of ROS and RNS which cannot be adequately dealt with by the endogenous antioxidant system can lead to damage of lipids, proteins, carbohydrates, and nucleic acids. Changes in oxidative metabolism which lead to an increase in the oxidizing environment and the formation of potentially physiologically-deleterious reactive oxygen species (ROS) and that of reactive nitrogen species (RNS) has been generally termed within the literature as “oxidative stress”. It has also recently been recognized that cancer cells may respond to such “oxidative stress”, induced by chemotherapy or radiation exposure, by decreasing the concentrations of ROS and oxidized thiols and well as by increased concentrations of thiol and anti-oxidants. It should be noted that when either or both of these mechanisms are operative, the subject's tumor cells may become resistant to chemotherapy and radiation therapy, thereby representing an important obstacle to curing or controlling the progression of the subject's cancer.

### II. Physiological Cellular Thiols

Thiol groups are those which contain functional CH₂—SH groups within conserved cysteinyl residues. It is these thiol-containing proteins which have been elucidated to play the primary role in redox-sensitive reactions. Their redox-sensing abilities are thought to occur by electron flow through the sulfhydryl side-chain. Thus, it is the unique properties afforded by the sulfur-based chemistry in protein cysteines (in some cases, possibly in conjunction with chelated central metal atoms) that is exploited by transcription factors which “switch” between an inactive and active state in response to elevated concentrations of ROS and/or RNS. It should be noted that the majority of cellular protein thiols are compartmentalized within highly reducing environments and are therefore “protected” from such oxidation. Hence, only proteins with accessible thiol moieties, and higher oxidation potentials are likely to be involved in redox-sensitive signaling mechanisms.

There are numerous naturally-occurring thiols and disulfides that are involved in oxidative metabolism. The most abundant biologically-occurring amino acid is cysteine, along with its disulfide form, cystine. Another important and highly abundant intracellular thiol is glutathione (GSH), which is a tripeptide comprised of γ-glutamate-cysteine-glycine. Thiols can also be formed in those amino acids which contain cysteine residues including, but not limited to, cystathionine, taurine, and homocysteine. Many oxidoreductases and transferases rely upon cysteine residues for their physiological catalytic functions. There are also a large number of low molecular weight cysteine-containing compounds, such a Co-enzyme A and glutathione, which are vital enzymes in maintaining oxidative/reductive homeostasis in cellular metabolism. These compounds may also be classified as non-protein sulfhydryls (NPSH).

Structural and biochemical data has also demonstrated that thiol-containing cysteine residues and the disulfide cystine, play a ubiquitous role in allowing proteins to respond to ROS. The redox-sensitivity of specific cysteine residues imparts specificity to ROS-mediated cellular signaling. By reacting with ROS, cysteine residues function as “detectors” of redox status; whereas the consequent chemical change in the oxidized cysteine can be converted into a protein conformational change, hence providing an activity or response.

Within biological systems, thiols undergo a reversible oxidation/reduction reaction, as illustrated below, which are often catalyzed by transition metals. These reactions can also involve free radicals (e.g., thioyl RS) as intermediates. In addition, proteins which possess SH/SS groups can interact with the reduced form of GSH in a thiol-disulfide exchange. Thiols and their disulfides are reversibly linked, via specific enzymes, to the oxidation and reduction of NADP and NADPH. This reversible oxidation/reduction reaction is shown in Table 1, below:

TABLE 1

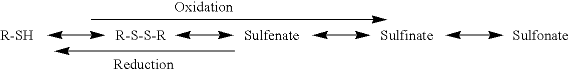

There is increasing experimental evidence that indicates that thiol-containing proteins are sensitive to thiol modification and oxidation when exposed to changes in the redox state. This sensing of the redox potential is thought to occur in a wide range of diverse signal transduction pathways. Moreover, these redox sensing proteins play roles in mediating cellular responses to changes in intracellular oxidative metabolism (e.g., increased cellular proliferation).

One of the primary enzymes involved in the synthesis of cellular thiols is cysteine synthase, which is widely distributed in human tissues, where it catalyzes the synthesis of cysteine from serine. The absorption of cystine and structurally-related amino acids (e.g., ornithine, arginine, and lysine) are mediated by a complex transporter system. The Xc transporter, as well as other enzymes, participate in these cellular uptake mechanisms. Once transported into the cell, cystine is rapidly reduced to cysteine, in an enzymatic reaction which utilizes reduced glutathione (GSH). In the extracellular environment, the concentrations of cystine are typically substantially higher than cysteine, and whereas the reverse is true in the intracellular environment.

### III. BNP7787 and Mesna Heteroconjugates

BNP7787 (disodium 2,3′-dithio-bis-ethansulfonate; dimesna; Tavocept™) is a water-soluble disulfide drug currently undergoing clinical development for, by way of non-limiting example, the prevention of chemotherapy-induced toxicities, including neurotoxicity caused by taxanes and platinum-based chemotherapy and the cumulative renal toxicity caused by cisplatin. See, e.g., Kelland, L. R., Meeting report on 8th international symposium on platinum and other metal coordination compounds in cancer chemotherapy. J. Inorg. Biochem. 77:121-124 (1999); Senior, K. Supercomputer-designed drug protects against chemotherapy toxicity. Lancet Oncol. 1:198 (2000).

BNP7787 has been shown to cause the reversible modulation of plasma thiol and disulfide levels, a property that may contribute to both its chemoprotective action on normal tissue and sensitization of tumors to chemotherapy. Previous studies have shown that BNP7787 distributes to the kidney, liver, intestine and marrow, but little has been reported regarding the intracellular pharmacology and metabolism of BNP7787. Pre-clinical studies have demonstrated that BNP7787 does not exhibit tumor protection in the presence of known chemotherapeutic agents (e.g., paclitaxel and cisplatin) and that BNP7787 pre-treatment can prevent lethal toxicity resulting from commonly utilized chemotherapy drugs including, but not limited to: paclitaxel, cisplatin, carboplatin, and oxaliplatin. See, e.g., Boven, E., et al., BNP7787, a novel protector against platinum-related toxicities, does not affect the efficacy of cisplatin or carboplatin in human tumour xenografts. Eur. J. Cancer 38:1148-1156 (2002); Pendyala, L., et al., Modulation of plasma thiols and mixed disulfides by BNP7787 in patients receiving paclitaxel/cisplatin therapy. Cancer Chemother. Pharmacol. 51:376-384 (2003); Verschraagen, M., et al., Pharmacokinetics and preliminary clinical data of the novel chemoprotectant BNP7787 and cisplatin and their metabolites. Clin. Pharmacol. Ther. 74:157-169 (2003). Additionally, recent BNP7787 Phase 2 and Phase 3 clinical studies have demonstrated a marked increase in the median survival time, as well as a decrease in chemotherapy-induced nephrotoxicity and emesis, in trial patients with non-small cell lung carcinoma, including the adenocarcinoma sub-type, as set forth in U.S. patent application Ser. Nos. 12/075,980 and 12/218,470, whose disclosures are incorporated by reference herein, in their entirety.

In the present invention, non-enzymatic thiol-disulfide exchange reactions that occur between BNP7787, its metabolite, mesna (sodium 2-mercaptoethane sulfonate; Mesnex®), and physiological thiols and disulfides are disclosed. The rates for formation of BNP7787 derived, mesna mixed-disulfides were calculated and the potential of the mesna heteroconjugates (i.e., mesna mixed disulfides) to serve as substrates for the thioredoxin (Trx) system are also disclosed. BNP7787 and BNP7787-derived mesna heteroconjugates are reduced by thioredoxin, but not by thioredoxin reductase (TrxR). The non-enzymatic thiol-disulfide exchange reactions of BNP7787 and mesna may have important roles in the metabolism of BNP7787 by the Trx system. A BNP7787-mediated shift towards oxidized (i.e., inactive) Trx, may partially contribute to increased survival benefits for non-small cell lung carcinoma, including the adenocarcinoma sub-type, patients that have been observed in multiple clinical trials. See, U.S. patent application Ser. Nos. 12/075,980 and 12/218,470, whose disclosures are incorporated by reference herein, in their entirety. Major obstacles to the treatment of human cancer involve the mediation of numerous factors that include, but are not limited to: (i) apoptotic resistance to chemotherapy; (ii) decoupled cellular proliferation signaling; (iii) enhance RNA to DNA conversion; (iv) increased transcription and translation and DNA biosynthesis; and (v) pro-angiogenic signaling. In addition, some newly elucidated, potential mechanisms for restoring apoptotic sensitivity by BNP7787 and its metabolites, are disclosed herein. In brief, BNP7787 appears to act by direct modulation of the intracellular balance of oxidized and reduced thioredoxin (Trx).

Preliminary drug uptake experiments in whole animals demonstrated that radiolabeled BNP7787 distributes to the kidney, liver, and intestinal epithelia (see, e.g., Ormstad, K. and Uehara, N., Renal transport and disposition of Na-2-mercaptoethane sulfonate disulfide (dimesna) in the rat. FEBS Lett. 150:354-358 (1982); Brock, N., et al., Pharmacokinetics and mechanism of action of detoxifying low-molecular-weight thiols. J. Cancer Res. Clin. Oncol. 108:87-97 (1984); Ormstad, K. and Ohno, Y., N-acetylcysteine and sodium 2-mercaptoethane sulfonate as sources of urinary thiol groups in the rat. Cancer Res. 44:3797-3800 (1984); Goren, M. P., et al., Reduction of dimesna to mesna by the isolated perfused rat liver. Cancer Res. 58:4358-4362 (1998); Hausheer, F. H., et al., New approaches to drug discovery and development: a mechanism-based approach to pharmaceutical research and its application to BNP7787, a novel chemoprotective agent. Cancer Chemother. Pharmacol. 52 (Suppl 1):S3-15 (2003)) and, that in the plasma, BNP7787 does not undergo any appreciable metabolism to its primary metabolite, mesna (see, e.g., Verschraagen, M., et al., Quantification of BNP7787 (dimesna) and its metabolite mesna in human plasma and urine by high-performance liquid chromatography with electrochemical detection. J. Chromatogr. B. Biomed. Sci. Appl. 753:293-302 (2001); Verschraagen, M., et al., Pharmacokinetics of BNP7787 and its metabolite mesna in plasma and ascites: A case report. Cancer Chemother. Pharmacol. 51:525-529 (2003); Verschraagen, M., et al., The chemical reactivity of BNP7787 and its metabolite mesna with the cytostatic agent cisplatin: comparison with the nucleophiles thiosulfate, DDTC, glutathione and its disulfide GSSG. Cancer Chemother. Pharmacol. 51:499-504 (2003)). Thus, it is possible that the primary site of BNP7787 metabolism occurs intracellularly or in the interstitial space, and this metabolism may involve non-enzymatic or enzymatic mechanisms. Accordingly, in the present application we propose that non-enzymatic thiol disulfide exchanges involving BNP7787, its metabolite, mesna, and physiological thiols and disulfides may account for a number of the therapeutic effects observed for BNP7787. The metabolism of BNP7787 to heterodisulfides of mesna has been supported by computational studies of thermodynamic probabilities of non-enzymatic thiol transfer reactions involving physiological free thiols with BNP7787. See, Hausheer, F. H., et al., New approaches to drug discovery and development: a mechanism-based approach to pharmaceutical research and its application to BNP7787, a novel chemoprotective agent. Cancer Chemother. Pharmacol. 52 (Suppl 1):S3-15 (2003) The metabolite of BNP7787 (i.e., the reduced form), sodium 2-mercapto-ethanesulfonate (mesna), is more chemically reactive and has the potential to participate in reactions with a number of physiological disulfides including glutathione disulfide (GSSG) and cystine. Id.

The present invention discloses the non-enzymatic formation of BNP7787-derived, mesna heteroconjugates and evaluates the interactions of BNP7787, mesna and the non-enzymatically-formed mesna heteroconjugates with thioredoxin (Trx) and thioredoxin reductase (TrxR). The thioredoxin (Trx) system along with the glutaredoxin (Grx) system comprise the main thiol redox buffering systems in mammalian cells. Both the Trx and Grx systems are composed of disulfide oxidoreductases and flavin-binding enzymes involved in disulfide exchange reactions among small molecules and proteins. While they share some substrate specificity, the Trx system is more catalytically diverse than the Grx system and does not interact substantially with glutathione, the preferred substrate of the Grx system.

The Trx system plays an important role in the redox regulation of a number of important cellular processes, including modulation of apoptotic and cellular proliferation pathways. The system includes the selenoprotein, thioredoxin reductase (TrxR), and its main substrate, thioredoxin (Trx), as well as thioredoxin peroxidase (TPX). See, e.g., Prieto-Alamo, M.-J., et al., Transcriptional regulation of glutaredoxin and thioredoxin pathways and related enzymes in response to oxidative stress. J. Biol. Chem. 275:13398-13405 (2000); Amer, E. S. J., et al., Physiological functions of thioredoxin and thioredoxin reductase. Eur. J. Biochem 267:6102-6109 (2000); Powis, G., et al., The role of the redox protein thioredoxin in cell growth and cancer. Free Rad. Biol. Med. 29:312-322 (2000). TrxR is a pyridine nucleotide-disulfide oxidoreductase that catalyzes the NADPH-dependent reduction of the active site disulfide in oxidized thioredoxin (Trx-S₂) to give a dithiol in reduced thioredoxin (Trx-(SH)₂). See, e.g., Reed, D. J., Molecular and Cellular Mechanisms of Toxicity (De Matteis, F. and Smith, L. L., eds.), pp. 35-68, CRC Press, Boca Raton (2002). TrxR also contains a selenocysteine group that can form a selenenylsulfide bond with another cysteine on the protein. See, e.g., Jacob, C., et al., H. Sulfur and Selenium: The role of oxidation state in protein structure and function. Angew. Chem. Int. Ed. 42:4742-4758, 2003. Trx is a small disulfide reductase that requires TrxR for catalytic cycling. Trx has a broad substrate specificity and important functions in the redox modulation of protein signaling and the reductive activation of transcription factors, including NF-κB, AP-1, and p53. Trx is only active in its reduced form (Trx-(SH)₂) which serves as a hydrogen donor for ribonucleotide reductase and other redox enzymes, and functions in defense against oxidative stress. The present invention discloses the characterization of the thiol disulfide exchanges that occur between BNP7787, mesna and physiologically-relevant thiols and disulfides and the evaluation of these aforementioned interactions of these compounds with thioredoxin (Trx) and thioredoxin reductase (TrxR). The potential of the disclosed compounds to modulate the intracellular balance of oxidized and reduced thioredoxin (Trx) may be involved in a variety of physiological processes including, but not limited to, a reduction or prevention of chemotherapy-induced side-effects.

### SUMMARY OF THE INVENTION

The invention described and claimed herein has many attributes and embodiments including, but not limited to, those set forth or described or referenced in this Summary section. However, it should be noted that this Summary is not intended to be all-inclusive, nor is the invention described and claimed herein limited to, or by, the features or embodiments identified in said Summary. Moreover, this Summary is included for purposes of illustration only, and not restriction.

The present invention discloses compounds that are useful to mitigate or prevent deleterious chemotherapy-induced, deleterious physiological side-effects in a patient with cancer who is receiving one or more chemotherapeutic agents. The compounds possess the following generic structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

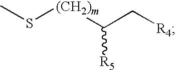

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

Another embodiment of the present invention discloses a method to modulate the intracellular balance of oxidized and reduced thioredoxin (Trx) in a patient with cancer who is receiving one or more chemotherapeutic agents, with the method comprising the administration of a medically-sufficient amount of a compound having the formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

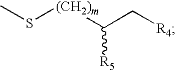

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

In another embodiment, the compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or

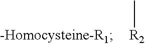

wherein R₁ and R₂ are any L- or D-amino acids; and pharmaceutically-acceptable salts and prodrugs thereof.

Another embodiment of the present invention discloses a kit comprising a compound for administration and instructions for administering the compound to a patient with cancer who is receiving one or more chemotherapeutic agents, in an amount sufficient to mitigate or prevent chemotherapy-induced, physiologically-deleterious side-effects, said kit comprising the administration of a medically-sufficient amount of a compound having the generic structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

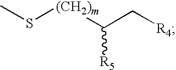

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

One embodiment of the present invention discloses a kit comprising a compound for administration and instructions for administering the compound to a patient with cancer who is receiving one or more chemotherapeutic agents, in an amount sufficient to modulate the intracellular balance of oxidized and reduced thioredoxin (Trx), said kit comprising the administration of a medically-sufficient amount of a compound having the generic structural formula: X—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

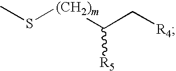

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids; or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

In other embodiments, the compound is in the form of a pharmaceutical formulations comprising one or more compounds possessing the structural formula: X—S—S—R₁—R₂, as the active agent, combined with one or more pharmaceutically-acceptable excipients, fillers, diluents or additives to produce a formulation which is suitable for administration to human patients.

In yet other embodiments, the compound is a sodium salt, a potassium salt, a calcium salt, a magnesium salt, an ammonium salt, or a manganese salt.

In other embodiments, the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

In still other embodiments, the chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

Various additional embodiments will become apparent upon a reading of the following description and claims.

### BRIEF DESCRIPTION OF THE FIGURES

FIG. 1: illustrates a preferred synthesis of the resin-bound mesna intermediates. The resin, preferably polystyrene microspheres of 200-400 mesh size, is functionalized with an appropriate linker, shown in FIG. 1 as sodium sulfinate.

FIG. 2: illustrates the synthesis of the compounds of the present invention where R₁ is ethyl and R₂ is sulfonate. As shown, the synthetic process is a one-step, single pot process in which the polymer bound mesna is reacted with a sulfur-containing amino acid, preferably cysteine, homocysteine or glutathione; or by a short-chain peptide having 2-10 amino acids, at least one of which is a sulfur-containing amino acid.

FIG. 3: illustrates the structures of BNP7787 (Tavocept), mesna, glutathione and selected BNP7787-derived mesna heteroconjugates. The mesna portion of the heteroconjugates is shaded in a gray box.

FIG. 4: illustrates, in graphical form, the EC HPLC analyses over time of reactions between A) BNP7787 and Cysteine; B) BNP7787 and homocysteine; C) BNP7787 and Glutathione; D) Mesna and Cystine; E) Mesna and Homocystine; and E) Mesna and glutathione disulfide. All compounds were 100 μM in concentration and were incubated in Buffer A, pH 7.4. Chromatographs were optimized to maximize the largest peak in each chromatograph and, therefore, due to varying responses of the various thiols and disulfides to the HPLC EC detector, the y-axes may vary.

FIG. 5: HPLC-UV Chromatograms of the Reaction Between BNP7787 (100 μM) and Cysteine (100 μM) in Buffer A (pH 7.4) at 37° C. Reactions between BNP7787 (100 μM) and either homocysteine (100 μM) or glutathione (100 μM) resulted in similar HPLC-UV chromatograms (i.e., disappearance of the BNP7787 peak) and are not shown.

FIG. 6: illustrates the kinetic schemes for the reactions with BNP7787 or mesna.

FIG. 7: A. illustrates BNP7787 and mesna heteroconjugates as substrates for the TrxR/Trx system. NADPH oxidation by TrxR and Trx (Table 3, Reaction B conditions) increases in the presence of increasing concentrations of BNP7787 (filled circles); GSSM (filled squares); CSSM (filled triangles); and HSSM (filled diamonds).
B. illustrates BNP7787 inhibits the TrxR/Trx catalyzed reduction of the insulin disulfide in a concentration dependent manner (10 mM BNP7787 inhibited the reaction essentially identically to 4 mM glutathione disulfide).

FIG. 8: illustrates, pictorially, a possible mechanism for BNP7787 (Tavocept) and BNP7787-derived mesna heteroconjugate modulation of the intracellular balance of oxidized (inactive) and reduced (active) thioredoxin (Trx).

### DESCRIPTION OF THE PREFERRED EMBODIMENTS

The preferred embodiments herein described are not intended to be exhaustive or to limit the invention to the precise form disclosed. They are chosen and described to explain the principles of the invention, and its application and practical use to thereby enable others skilled in the art to follow its teachings.

### DEFINITIONS

As utilized herein, the term “generic structural formula” refers to the fixed structural part of the molecule of the formula given.

“Lower alkylene” means a bridging moiety formed of one to six ‘—CH₂—’ groups.

“Aryl” means an aromatic ring or ring system consisting of one or more rings, preferably one to three rings, fused or unfused, with the ring atoms consisting entirely of carbon atoms.

“Lower alkyl” means a straight or branched-chain aliphatic hydrocarbon containing one to six carbon atoms.

“Lower alkenyl” and “lower alkynyl” means a straight or branched chain hydrocarbon containing one to six carbon atoms, and with at least one double bond (alkenyl) or triple bond (alkynyl) between two of the carbon atoms.

As utilized herein, the terms “compounds of the present invention” and “novel compounds of the present invention” mean compounds having the generic structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

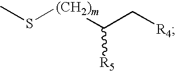

R₂ and R₄ is sulfonate or phosphonate;
R₁ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.
   It should be noted that the aforementioned terms are also inclusive of the terms “mesna heteroconjugate”, “mesna conjugate”, or “mesna derivative”, and may be utilized interchangeably, herein,

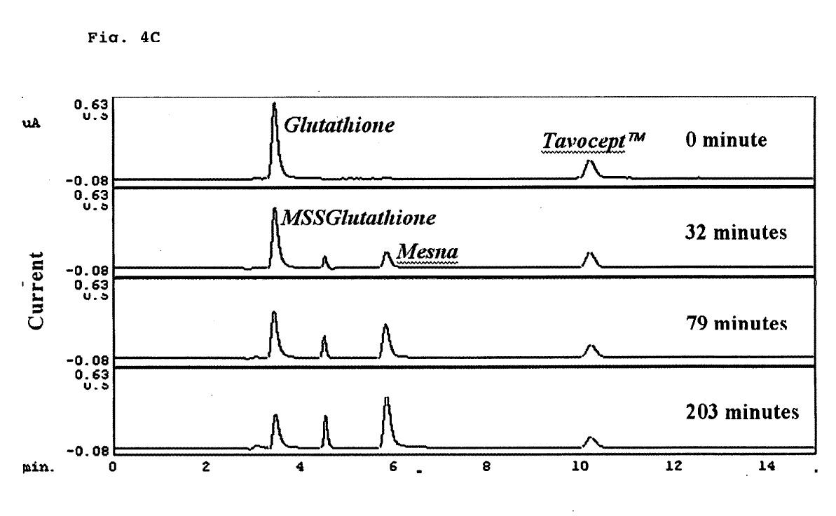

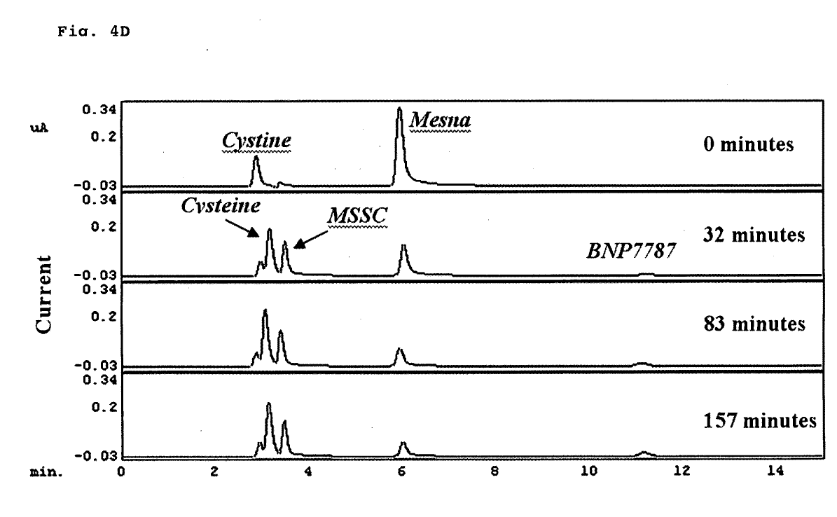

As used herein, the terms “mesna heteroconjugate”, “mesna conjugate”, or “mesna derivative” represent the metabolite of disodium 2,2′-dithio-bis-ethane sulfonate, known as 2-mercapto ethane sulfonate sodium (mesna) as a disulfide form which is conjugated with a substituent group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or -

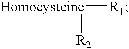

wherein R₁ and R₂ are any L- or D-amino acids. Various mesna heteroconjugates of the present invention are illustrated in FIG. 3, wherein the mesna portion of the molecule is shown by grey highlighting.

As utilized herein the terms “fragments”, “moieties” or “substituent groups” are the variable parts of the molecule, designated in the formula by variable symbols, such as Rₓ, X or other symbols. Substituent Groups may consist of one or more of the following:

As utilized herein, the definitions for the terms “adverse event” (effect or experience), “adverse reaction”, “unexpected adverse reaction” and “side-effect” have previously been agreed to by consensus of the more than thirty Collaborating Centers of the WHO International Drug Monitoring Centre (Uppsala, Sweden). See, Edwards, I. R., et al., Harmonisation in Pharmacovigilance Drug Safety 10 (2):93-102 (1994). The following definitions, with input from the WHO Collaborative Centre, have been agreed to:

1. Adverse Event (Adverse Effect or Adverse Experience)—Any untoward medical occurrence in a patient or clinical investigation subject administered a pharmaceutical product and which does not necessarily have to have a causal relationship with this treatment. An Adverse Event (AE) can therefore be any unfavorable and unintended sign (including an abnormal laboratory finding, for example), symptom, or disease temporally associated with the use of a medicinal product, whether or not considered related to the medicinal product.

2. Adverse Drug Reaction (ADR)—In the pre-approval clinical experience with a new medicinal product or its new usages, particularly as the therapeutic dose(s) may not be established: all noxious and unintended responses to a medicinal product related to any dose should be considered adverse drug reactions. Drug-related Adverse Events are rated from grade 1 to grade 5 and relate to the severity or intensity of the event. Grade 1 is mild, grade 2 is moderate, grade 3 is severe, grade 4 is life threatening, and grade 5 results in death.

3. Unexpected Adverse Drug Reaction—An adverse reaction, the nature or severity of which is not consistent with the applicable product information.

Serious Adverse Event or Adverse Drug Reaction: A Serious Adverse Event (experience or reaction) is any untoward medical occurrence that at any dose:
(a) Results in death or is life-threatening. It should be noted that the term “life-threatening” in the definition of “serious” refers to an event in which the patient was at risk of death at the time of the event; it does not refer to an event which hypothetically might have caused death if it were more severe.
(b) Requires inpatient hospitalization or prolongation of existing hospitalization.
(c) Results in persistent or significant disability/incapacity, or
(d) Is a congenital anomaly/birth defect.

As utilized herein, the terms “chemotherapy-induced side-effects” or “chemotherapy-induced, physiologically-deleterious side-effects” includes, but is not limited to side-effects that include: neurotoxicity such as peripheral neuropathy (both cumulative and sporadic/intermittent), nephrotoxicity, ototoxicity, allergic or hypersensitivity reactions, hepatic toxicity, myelosuppression, anemia, nausea, emesis, as well as other toxicities.

As utilized herein the term “cancer” refers to all known forms of cancer including, solid forms of cancer (e.g., tumors), lymphomas, and leukemias.

As utilized herein, the term “adenocarcinoma” refers to a cancer that originates in glandular tissue. Glandular tissue comprises organs that synthesize a substance for release such as hormones. Glands can be divided into two general groups: (i) endocrine glands—glands that secrete their product directly onto a surface rather than through a duct, often into the blood stream and (ii) exocrine glands—glands that secrete their products via a duct, often into cavities inside the body or its outer surface. However, it should be noted that to be classified as adenocarcinoma, the tissues or cells do not necessarily need to be part of a gland, as long as they have secretory properties. Adenocarcinoma may be derived from various tissues including, but not limited to, breast, colon, lung, prostate, salivary gland, stomach, liver, gall bladder, pancreas (99% of pancreatic cancers are ductal adenocarcinomas), cervix, vagina, and uterus, as well as unknown primary adenocarcinomas. Adenocarcinoma is a neoplasm which frequently presents marked difficulty in differentiating from where and from which type of glandular tissue the tumor(s) arose. Thus, an adenocarcinoma identified in the lung may have had its origins (or may have metastasized) from an ovarian adenocarcinoma. Cancer for which a primary site cannot be found is called cancer of unknown primary.

As utilized herein, the term “non-small cell lung cancer (NSCLC)” accounts for approximately 75% of all primary lung cancers. NSCLC is pathologically characterized further into adenocarcinoma, squamous cell carcinoma, large cell carcinoma, and various other less common forms.

As utilized herein, the terms “chemotherapy” or “chemotherapeutic regimen(s)” or “chemotherapy cycle” refer to treatment using the above-mentioned chemotherapeutic agents with or without the use of an oxidative metabolism-affecting compound of the present invention.

As used herein, the term “chemotherapeutic agent” or “chemotherapy agent” or “chemotherapeutic drug” refer to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include, for example, fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

As utilized herein, the terms “chemotherapy”, “chemotherapeutic regimen(s)”, or “chemotherapy cycle” refer to treatment using the above-mentioned chemotherapeutic agents with or without the novel compounds of the present invention.

As used herein, the term “platinum medicaments” or “platinum compounds” include all compounds, compositions, and formulations which contain a platinum ligand in the structure of the molecule. By way of non-limiting example, the valence of the platinum ligand contained therein may be platinum II or platinum IV. The platinum medicaments or platinum compounds of the present invention include, in a non-limiting manner, cisplatin, oxaliplatin, carboplatin, satraplatin, and analogs and derivatives thereof.

As used herein, the term “taxane medicaments” include, in a non-limiting manner, docetaxel or paclitaxel (including the commercially-available paclitaxel derivatives Taxol® and Abraxane®), polyglutamylated forms of paclitaxel (e.g., Xyotax®), liposomal paclitaxel (e.g., Tocosol®), and analogs and derivatives thereof.

As utilized herein, the term “oxidative metabolism-affecting compound” is a compound, formulation, or agent which is capable of: mitigating or preventing: (i) the overexpression (or increased activity, or both) of thioredoxin or glutaredoxin in cancer cells; (ii) the loss of apoptotic sensitivity to therapy (i.e., drug or ionizing radiation resistance); (iii) increased conversion of RNA into DNA (involving ribonucleotide reductase); (iv) altered gene expression; (v) increased cellular proliferation signals and rates; (vi) increased thioredoxin peroxidase; and/or (vii) increased angiogenic activity (i.e., increased blood supply to the tumor). Accordingly, by pharmacological inactivation or modulation of thioredoxin and/or glutaredoxin by the proper medical administration of effective levels and schedules of the oxidative metabolism-affecting compounds of the present invention, can result in enhancement of chemotherapy effects and thereby lead to increased patient survival.

As used herein, a “medically-sufficient dose” or a “medically-sufficient amount” in reference to the compounds or compositions of the instant invention refers to the dosage that is sufficient to induce a desired biological, pharmacological, or therapeutic outcome in a subject with neoplastic disease. That result can be: (i) cure or remission of previously observed cancer(s); (ii) shrinkage of tumor size; (iii) reduction in the number of tumors; (iv) delay or prevention in the growth or reappearance of cancer; (v) selectively sensitizing cancer cells to the anti-cancer activity of chemotherapeutic agents; (vi) restoring or increasing apoptotic effects or sensitivity in tumor cells; and/or (vii) increasing the time of survival of the patient, alone or while concurrently experiencing reduction, prevention, mitigation, delay, shortening the time to resolution of, alleviation of the signs or symptoms of the incidence or occurrence of an expected side-effect(s), toxicity, disorder or condition, or any other untoward alteration in the patient.

As utilized herein, the term “patient” refers to any individual or subject, without limitation, who is in need of treatment with a compound, composition, medicament, formulation, method, or kit which is disclosed in the present invention.

As used herein, the term “pharmaceutically-acceptable salt” means salt derivatives of drugs which are accepted as safe for human administration. In the present invention, the compounds of the present invention include pharmaceutically-acceptable salts, which include but are not limited to: (i) a monosodium salt; (ii) a calcium salt; (iii) a magnesium salt; (iv) a manganese salt; (v) an ammonium salt; and (vi) a monopotassium salt.

As used herein the terms “reactive oxygen species (ROS)” and “reactive nitrogen species (RNS)” refer to ionic species which may result from a variety of metabolic and/or environmental processes. By way of non-limiting example, intracellular ROS (e.g., hydrogen peroxide: H₂O₂, superoxide anion: O₂⁻, hydroxyl radical: OH⁻, nitric oxide, and the like) may be generated by several mechanisms: (i) by the activity of radiation; (ii) during xenobiotic and drug metabolism; and (iii) under relative hypoxic, ischemic and catabolic metabolic conditions.

As used herein, the term “reducing” includes preventing, attenuating the overall severity of, delaying the initial onset of, and/or expediting the resolution of the acute and/or chronic pathophysiology associated with malignancy in a subject.

As used herein the term “redox state”, “redox potential”, “oxidative/reductive state” of any particular biological environment can be defined as the sum of oxidative and reductive processes occurring within that environment, which affects the extent to which molecules are oxidized or reduced within it. The redox potential of biological ions or molecules is a measure of their tendency to lose an electron (i.e., thereby becoming oxidized). Under normal physiological circumstances, most intracellular biological systems are predominantly found in a reduced state. Within cells, thiols (R—SH) such as glutathione (GSH) are maintained in their reduced state, as are the nicotinamide nucleotide coenzymes NADH and NADPH. Conversely, plasma is generally an oxidizing environment due to the high partial pressure of oxygen and the relative absence of disulfide reducing enzymes. Physiological circumstances can, however, arise which alter the overall redox balance and lead to a more oxidizing environment on cells. In biological systems, this activity arises as a result of changes in intracellular oxidative metabolism and physiological systems have evolved to preserve, protect, and control the normal reducing environment. However, when the changes overwhelm these protective mechanisms, oxidative damage and profound biological changes can occur. Cancer cells have been observed to have the ability to mount more effective anti-oxidative responses to changes in intracellular oxidative metabolism (e.g., oxidative stress) in comparison to normal, non-cancerous, cells, thereby leading to a survival advantage and the ability to resist or escape the anti-cancer and cytotoxic action of chemotherapeutic agent(s).

As utilized herein, the term “redox response” refers to the biological response to induce antioxidant systems against changes in oxidative metabolism to maintain the homeostasis in the intracellular redox balance.

As used herein, the term “receive” or “received” refers to a subject who has cancer and who has received, is currently receiving, or will receive one or more chemotherapeutic agents and/or an oxidative metabolism-affecting Formula (I) compound of the present invention.

The term “solvate” or “solvates” refers to a molecular complex of a compound such as an oxidative metabolism-affecting compound of the present invention with one or more solvent molecules. Such solvent molecules are those commonly used in the pharmaceutical art (e.g., water, ethanol, and the like). The term “hydrate” refers to the complex where the solvent molecule is water.

As used herein, the term “treat” or “treated”, with respect to a patient without cancer, refers to a patient, who is in need thereof, and who has received, is currently receiving, or will receive novel compound(s) of the present invention.

As used herein, the term “treat” or “treated”, with respect to a patient with cancer, refers to a patient who has received, is currently receiving, or will receive one or more chemotherapeutic agents and/or novel compound(s) of the present invention.

As used herein, “treatment schedule time” or “treatment regimen” means the difference in schedule of administration time, including: (i) the amount of drug administered per day or week; (ii) the amount of drug administered per day or week per m² of body surface area; or (iii) the amount of drug administered per day or week per kg of body weight.

### I. The Thioredoxin Reductase (TrxR)/Thioredoxin (Trx) System

### Thioredoxin Reductase (TrxR)

The thioredoxin system is comprised of thioredoxin reductase (TrxR) and its main protein substrate, thioredoxin (Trx), where the catalytic site disulfide of Trx is reduced to a dithiol by TrxR at the expense of NADPH. The thioredoxin system, together with the glutathione system (comprising NADPH, the flavoprotein glutathione reductase, glutathione, and glutaredoxin), is regarded as a main regulator of the intracellular redox environment, exercising control of the cellular redox state and antioxidant defense, as well as governing the redox regulation of several cellular processes. The system is involved in direct regulation of: (i) several transcription factors, (ii) apoptosis (i.e., programmed cell death) induction, and (iii) many metabolic pathways (e.g., DNA synthesis, glucose metabolism, selenium metabolism, and vitamin C recycling). See, e.g., Amér, E. S. J., et al., Physiological functions of thioredoxin and thioredoxin reductase. Eur. J. Biochem. 267:6102-6109 (2000). In addition to Trx, other endogenous substrates have been demonstrated for TrxR including, but not limited to, lipoic acid; lipid hydroperoxides; the cytotoxic peptide NK-lysin; vitamin K; dehydroascorbic acid; the ascorbyl free radical; and the tumor-suppressor protein p53. See, e.g., Reed, D. J., Molecular and Cellular Mechanisms of Toxicity (DeMatteis, F. and Smith, L. L., eds.), pp. 35-68, CRC Press, Boca Raton (2002). However, the exact physiological role that TrxRs play in the reduction of most of these substrates has not yet been fully defined.

The mammalian thioredoxin reductases (TxrRs) are enzymes belonging to the avoprotein family of pyridine nucleotide-disulfide oxidoreductases that includes lipoamide dehydrogenase, glutathione reductase, and mercuric ion reductase. Members of this family are homodimeric proteins in which each monomer includes an FAD prosthetic group, an NADPH binding site and an active site containing a redox-active disulfide. Electrons are transferred from NADPH via FAD to the active-site disulfide of TrxR, which then reduces the substrate. See, e.g., Williams, C. H., Chemistry and Biochemistry of Flavoenzymes (Muller, F., ed.), pp. 121-211, CRC Press, Boca Raton (1995).

TrxRs are named for their ability to reduce oxidized thioredoxins (Trxs), a group of small, ubiquitous redox-active peptides that undergoes reversible oxidation/reduction of two conserved cysteine (Cys) residues within the catalytic site. The mammalian TrxRs are selenium-containing flavoproteins that possess: (i) a conserved -Cys-Val-Asn-Val-Gly-Cys-catalytic site; (ii) an NADPH binding site; and (iii) a C-terminal Cys-Selenocysteine sequence that communicates with the catalytic site and is essential for its redox activity. See, e.g., Powis, G. and Monofort, W. R. Properties and biological activities of thioredoxins. Ann. Rev. Pharmacol. Toxicol. 41:261-295 (2001). These proteins exist as homodimers and undergo reversible oxidation/reduction. The activity of TrxR is regulated by NADPH, which in turn is produced by glucose-6-phosphate dehydrogenase (G6DP), the rate-limiting enzyme of the oxidative hexose monophosphate shunt (HMPS; also known as the pentose phosphate pathway). Two human TrxR isozyme genes have been cloned: a 54 Kda enzyme that is found predominantly in the cytoplasm (TrxR-1) and a 56 Kda enzyme that contains a mitochondrial import sequence (Trx-2). Id. A third isoform of TrxR, designated (TGR) is a Trx and glutathione reductase localized mainly in the testis, has also been identified. See, e.g., Sun, Q. A., et al., Selenoprotein oxidoreductase with specificity for thioredoxin and glutathione systems. Proc. Natl. Acad. Sci. USA 98:3673-3678 (2001). Additionally, both mammalian cytosolic TrxR-1 and mitochondrial TrxR-2 have alternative splice variants. In humans, five different 5′ cDNA variants have been reported. One of the splicing variants exhibits a 67 kDa protein with an N-terminal elongation instead of the common 55 kDa. The physiological functions of these TrxR splice variants have yet to be elucidated. See, e.g., Sun, Q. A., et al., Heterogeneity within mammalian thioredoxin reductases: evidence for alternative exon splicing. J. Biol. Chem. 276:3106-3114 (2001).

The TrxR-1 isozyme has been the most extensively studied. TrxR-1, as purified from tissues such as placenta, liver, or thymus, and expressed in recombinant form, possesses wide substrate specificity and generally high reactivity with electrophilic agents. The catalytic site of TrxR-1 encompasses an easily accessible selenocysteine (Sec) residue situated within a C-terminal motif -Gly-Cys-Sec-Gly-COOH. See, e.g., Zhong, L., et al., Rat and calf thioredoxin reductase are homologous to glutathione reductase with a carboxyl-terminal elongation containing a conserved catalytically active penultimate selenocysteine residue. J. Biol. Chem. 273:8581-8591 (1998). Together with the neighboring cysteine, it forms a redox-active selenenylsulfide/selenolthiol motif that receives electrons from a redox-active-Cys-Val-Asn-Val-Gly-Cys-motif present in the N-terminal domain of the other subunit in the dimeric enzyme. See, e.g., Sandalova, T., et al., Three-dimensional structure of a mammalian thioredoxin reductase: implications for mechanism and evolution of a selenocysteine-dependent enzyme. Proc. Natl. Acad. Sci. USA 98:9533-9538 (2001). Substrates of the TrxR-1 enzyme, that can be reduced by the selenolthiol motif, include: protein disulfides such as those in thioredoxin; NK-lysin; protein disulfide isomerase; calcium-binding proteins-1 and -2; and plasma glutathione peroxidase; as well as small molecules such as 5,5′-dithiobis(2-nitrobenzoate) (DTNB); alloxan; selenodiglutathione; methylseleninate; S-nitrosoglutathione; ebselen; dehydroascorbate; and alkyl hydroperoxides. See, e.g., Amk, E. S., et al., Preparation and assay of mammalian thioredoxin and thioredoxin reductase. Method. Enzymol. 300:226-239 (1999). Additionally, several quinone compounds can be reduced by the enzyme and one-electron reduced species of the quinones may furthermore derivatize the selenolthiol motif, thereby inhibiting the enzyme. The highly accessible selenenylsulfide/selenolthiol motif of the enzyme is extraordinarily reactive and can be rapidly derivatized by various electrophilic compounds.

Due to the many important functions of TrxR, it is not surprising that its inhibition could be deleterious to cells due to an inhibition of the whole thioredoxin system. Moreover, in addition to a general inhibition of the thioredoxin system as a mechanism for cytotoxicity, it has also been shown that selenium-compromised forms of TrxR may directly induce apoptosis in cells by a gain of function. See, e.g., Anestal, K., et al., Rapid induction of cell death by selenium-compromised thioredoxin reductase 1, but not by the fully active enzyme containing selenocysteine. J. Biol. Chem. 278:15966-15672 (2003). The signaling mechanisms of this apoptotic induction have not been presently elucidated. It is clear, however, that electrophilic compounds inhibiting TrxR may have significant cellular toxicity as a result of these effects. From these findings it may surmised that TrxR inhibition may be regarded as a potentially important mechanism by which several alkylating agents and various chemotherapeutic agents (e.g., the monohydrated complex of cisplatin, oxaliplatin, etc.) commonly utilized in anticancer treatment, may exert their cytotoxic effects.

### Thioredoxin (Trx)

Thioredoxins (Trxs) are proteins that act as antioxidants by facilitating the reduction of other proteins by cysteine thiol-disulfide exchange. While glutaredoxins mostly reduce mixed disulfides containing glutathione, thioredoxins are involved in the maintenance of protein sulfhydryls in their reduced state via disulfide bond reduction. See, e.g., Print, W. A., et al., The role of the thioredoxin and glutaredoxin pathways in reducing protein disulfide bonds in the Escherichia coli cytoplasm. J. Biol. Chem. 272:15661-15667 (1996). Thiol-disulfide exchange is a chemical reaction in which a thiolate group (S⁻) attacks a sulfur atom of a disulfide bond (—S—S—). The original disulfide bond is broken, and its other sulfur atom is released as a new thiolate, thus carrying away the negative charge. Meanwhile, a new disulfide bond forms between the attacking thiolate and the original sulfur atom. The transition state of the reaction is a linear arrangement of the three sulfur atoms, in which the charge of the attacking thiolate is shared equally. The protonated thiol form (—SH) is unreactive (i.e., thiols cannot attack disulfide bonds, only thiolates). In accord, thiol-disulfide exchange is inhibited at low pH (typically, <8) where the protonated thiol form is favored relative to the deprotonated thiolate form. The pKₐ of a typical thiol group is approximately 8.3, although this value can vary as a function of the environment. See, e.g., Gilbert, H. F., Molecular and cellular aspects of thiol-disulfide exchange. Adv. Enzymol. 63:69-172 (1990); Gilbert, H. F., Thiol/disulfide exchange equilibria and disulfide bond stability. Meth. Enzymol. 251:8-28 (1995).

Thiol-disulfide exchange is the principal reaction by which disulfide bonds are formed and rearranged within a protein. The rearrangement of disulfide bonds within a protein generally occurs via intra-protein thiol-disulfide exchange reactions; a thiolate group of a cysteine residue attacks one of the protein's own disulfide bonds. This process of disulfide rearrangement (known as disulfide shuffling) does not change the number of disulfide bonds within a protein, merely their location (i.e., which cysteines are actually bonded). Disulfide reshuffling is generally much faster than oxidation/reduction reactions, which actually change the total number of disulfide bonds within a protein. The oxidation and reduction of protein disulfide bonds in vitro also generally occurs via thiol-disulfide exchange reactions. Typically, the thiolate of a redox reagent such as glutathione or dithiothreitol (DTT) attacks the disulfide bond on a protein forming a mixed disulfide bond between the protein and the reagent. This mixed disulfide bond when attacked by another thiolate from the reagent, leaves the cysteine oxidized. In effect, the disulfide bond is transferred from the protein to the reagent in two steps, both thiol-disulfide exchange reactions.

Thioredoxin (Trx) was originally described in 1964 as a hydrogen donor for ribonucleotide reductase which is an essential enzyme for DNA synthesis in Escherichia coli. Human thioredoxin was originally cloned as a cytokine-like factor named adult T cell leukemia (ATL)-derived factor (ADF), which was first defined as an IL-2 receptor α-chain (IL-2Ra, CD25)-inducing factor purified from the supernatant of human T cell leukemia virus type-1 (HTLV-1)-transformed T cell ATL2 cells. See, e.g., Yordi, J., et al., ADF, a growth-promoting factor derived from adult T cell leukemia and homologous to thioredoxin: possible involvement of dithiol-reduction in the IL-2 receptor induction. EMBO J. 8:757-764 (1989).

Proteins sharing the highly conserved -Cys-Xxx-Xxx-Cys- and possessing similar three-dimensional structure (i.e., the thioredoxin fold) are classified as belonging to the thioredoxin family. In the cytosol, members of the thioredoxin family include: the “classical cytosolic” thioredoxin 1 (Trx-1) and glutaredoxin 1. In the mitochondria, family members include: mitochondrial-specific thioredoxin 2 (Trx-2) and glutaredoxin 2. Thioredoxin family members in the endoplasmic reticulum (ER) include: protein disulfide isomerase (PDI); calcium-binding protein 1 (CaBP1); ERp72; Trx-related transmembrane protein (TMX); ERdj5; and similar proteins. Macrophage migration inhibitory factor (MIF) is a pro-inflammatory cytokine which was originally described as a soluble factor expressed by activated T cells in delayed-type hypersensitivity. See, e.g., Morand, E. F., et al., MIF: a new cytokine link between rheumatoid arthritis and atherosclerosis. Nat. Rev. Drug Discov. 5:399-411 (2006). MIF also possesses a redox-active catalytic site and exhibits disulfide reductase activity. See, e.g., Kleeman, R., et al., Disulfide analysis reveals a role for macrophage migration inhibitory factor (MIF) as thiol-protein oxidoreductase. J. Mol. Biol. 280:85-102 (1998). MIF has pro-inflammatory functions, whereas thioredoxin 1 (Trx-1) exhibits both anti-inflammatory and anti-apoptotic functions. Trx-1 and MIF control their expression reciprocally, which may explain their opposite functions. However, Trx-1 and MIF also share various similar characteristics. For example, both have a similar molecular weight of approximately 12 kDa and are secreted by a leaderless export pathway. They both share the same interacting protein such as Jun activation domain-binding protein 1 (JABI) in cells. Glycosylation inhibitory factor (GIF), which was originally reported as a suppressive factor for IgE response, is a posttranslationally-modified MIF with cysteinylation at Cys⁶⁰. The biological difference between MIF and GIF may be explained by redox-dependent modification, possibly involving TX-1. See, e.g., Nakamura, H., Thioredoxin and its related molecules: update 2005. Antioxid. Redox Signal. 7:823-828 (2005).

The mammalian thioredoxins (Trxs) are a family of 10-12 Kda proteins that contain a highly conserved -Trp-Cys-Gly-Pro-Cys-Lys- catalytic site. See, e.g., Nishinaka, Y., et al., Redox control of cellular functions by thioredoxin: A new therapeutic direction in host defense. Arch. Immunol. Ther. Exp. 49:285-292 (2001). The active site sequences is conserved from Escherichia coli to humans. Thioredoxins in mammalian cells possess >90% homology and have approximately 27% overall homology to the E. coli protein.

As previously discussed, the thioredoxins act as oxidoreductases and undergo reversible oxidation/reduction of the two catalytic site cysteine (Cys) amino acid residues. The most prevalent thioredoxin, Trx-1, is involved in a plethora of diverse biological activities. The reduced dithiol form of Trx [Trx-(SH)₂] reduces oxidized protein substrates that generally contain a disulfide group; whereas the oxidized disulfide form of Trx [Trx-(SS)] redox cycles back in an NADPH-dependent process mediated by thioredoxin reductase (TrxR), a homodimer comprised of two identical subunits each having a molecular weight of approximately 55 kDa. The conversion of thioredoxin from the disulfide form (oxidized) to the dithiol form (reduced) is illustrated in the diagram, below:

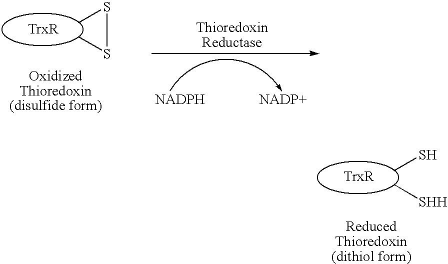

Two principal forms of thioredoxin (Trx) have been cloned. Trx-1 is a 105-amino acid protein. In almost all (>99%) of the human form of TX-1, the first methionine (Met) residue is removed by an N-terminus excision process (see, e.g., Giglione, C., et al., Protein N-terminal methionine excision. Cell. Mol. Life. Sci. 61:1455-1474 (2004), and therefore the mature protein is comprised of a total of 104 amino acid residues from the N-terminal valine (Val) residue. Trx-1 is typically localized in the cytoplasm, but it has also been identified in the nucleus of normal endometrial stromal cells, tumor cells, and primary solid tumors. Various types of post-translational modification of Trx-1 have been reported: (i) C-terminal truncated Trx-1, comprised of 1-80 or 1-84 N-terminal amino acids, is secreted from cells and exhibits more cytokine-like functions than full-length Trx-1; (ii) S-Nitrosylation at Cys⁶⁹ is important for anti-apoptotic effects; (iii) glutathionylation occurs at Cys⁷³, which is also the site responsible for the dimerization induced by oxidation; (iv) in addition to the original active site between Cys³²Cys³⁵, another dithiol/disulfide exchange is observed between and Cys⁶² and Cys⁶⁹, allowing intramolecular disulfide formation; and (v) Cys³⁵ and Cys⁶⁹ are reported to be the target for 15-deoxyprostaglandin-J₂. See, e.g., Nakamura, H., Thioredoxin and its related molecules: update 2005. Antioxid. Redox Signal. 7:823-828 (2005).

Reduced Trx-1, but not its oxidized form or a Cys→Ser catalytic site mutant, has been shown to bind to various intracellular proteins and may regulate their biological activities. In addition to NK-κB and Ref-1, Trx-1 binds to various isoforms of protein kinase C (PKC); p40 phagocyte oxidase; the nuclear glucocorticoid receptor; and lipocalin. Trx-1 also binds to apoptosis signal-regulating kinase 1 (ASK 1) in the cytosol under normal physiological conditions. However, when Trx-1 becomes oxidized under oxidative stress, ASK 1 is dissociated from Trx-1, and Trx-1 becomes a homodimer to transduce the apoptotic signal. ASK 1 is an activator of the JNK and p38 MAP kinase pathways, and is required for TNFα-mediated apoptosis. See, e.g., Saitoh, M., et al., Mammalian thioredoxin is a direct inhibitor of apoptosis signal-regulating kinase 1 (ask1). EMBO J. 17:2596-2606 (1998).

Another binding protein for Trx-1 is thioredoxin-binding protein 2 (TBP-2) which is identical to Vitamin D₃ upregulating protein 1 (VDUP1). TBP-2/VDUP1 was originally reported as the product of a gene whose expression was upregulated in HL-60 cells stimulated with la, 25-dihydroxyvitamin D₃. The interaction of TBP-2/VDUP1 with TrxR was observed both in vitro and in vivo. TBP-2/VDUP1 only binds to the reduced form of TrxR and acts as an apparent negative regulator of TrxR. See, e.g., Nishiyama, A., et al., Identification of thioredoxin-binding protein-2/Vitamin D(3) up-regulated protein 1 as a negative regulator of thioredoxin function and expression. J. Biol. Chem. 274:21645-21650 (1999). Although the mechanism is unknown, a reciprocal expression pattern of TrxR and TBP-2 was often reported upon various types of stimulation. Several highly homologous genes of TBP-2/VDUP1 have been identified. A TBP-2 homologue, TBP-2-like inducible membrane protein (TLIMP) is a novel VD3 or peroxisome proliferator-activated receptor-γ (PPAR-γ) ligand-inducible membrane-associated protein and plays a regulatory role in cell proliferation and PPAR-γ activation. See, e.g., Oka, S., et al., Thioredoxin-binding protein 2-like inducible membrane protein is a novel Vitamin D₃ and peroxisome proliferator-activated receptor (PPAR) gamma ligand target protein that regulates PPAR gamma signaling. Endocrinology 147:733-743 (2006). Another TBP-2 homologous gene, DRH1, is reported to be down-regulated in hepatocellular carcinoma. See, e.g., Yamamoto, Y., et al., Cloning and characterization of a novel gene, DRH1, down-regulated in advanced human hepatocellular carcinoma. Clin. Cancer Res. 7:297-303 (2001). These results indicate that the familial members of TBP-2 may also play a role in cancer suppression.

TBP-2 also possesses a growth suppressive activity. Overexpression of TBP-2 was shown to resulted in growth suppression. TBP-2 expression is upregulated by Vitamin D₃ treatment and serum- or IL-2-deprivation, thus leading to growth arrest. TBP-2 is found predominantly in the nucleus. TBP-2 mRNA expression is down-regulated in several tumors (see, e.g., Butler, L. M., et al., The histone deacetylase inhibitor SAHA arrests cancer cell growth, up-regulates thioredoxin-binding protein-2 and down-regulates thioredoxin. Proc. Natl. Acad. Sci. USA 99:11700-11705 (2002)) and lymphoma (see, e.g., Tome, M. E., et al., A redox signature score identifies diffuse large B-cell lymphoma patients with poor prognosis. Blood 106:3594-3601 (2005)), suggesting a close association between the expression reduction and tumorigenesis. TBP-2 expression is also downregulated in melanoma metastasis. See, e.g., Goldberg, S. F., et al., Melanoma metastasis suppression by chromosome 6: evidence for a pathway regulated by CRSP3 and TXNIP. Cancer Res. 63:432-440 (2003).

Loss of TBP-2 seems to be an important step of human T cell leukemia virus 1 (HTLV-1) transformation. In an in vitro model, HTLV-1-infected T-cells required IL-2 to proliferate in the early phase of transformation, but subsequently lost cell cycle control in the late phase, as indicated by their continuous proliferative state in the absence of IL-2. The change of cell growth phenotype has been suggested to be one of the oncogenic transformation processes. See, e.g., Maeda, M., et al., Evidence for the interleukin-2-dependent expansion of leukemic cells in adult T cell leukemia. Blood 70:1407-1411 (1987). The expression of TBP-2 is lost in HTLV-I-positive IL-2-independent T cell lines (due to the DNA methylation and histone deacetylation); but is maintained in HTLV-I-positive IL-2-dependent T cell lines, as well as in HTLV-1-negative T cell lines. See, e.g., Ahsan, M. K., et al., Loss of interleukin-2-dependancy in HTLV-1-infected T cells on gene silencing of thioredoxin-binding protein-2. Oncogene 25:2181-2191 (2005). Additionally, the murine knock-out HcB-19 strain, which has a spontaneous mutation in TBP-2/Txnip/VDUP1 gene, has been reported to have an increased incidence of hepatocellular carcinoma (HCC), showing that TBP-2/VDUP1 is a potential tumor suppressor gene candidate, in vivo. See, e.g., Sheth, S. S., et al., Thioredoxin-interacting protein deficiency disrupts the fasting-feeding metabolic transition. J. Lipid Res. 46:123-134 (2005). The same HcB-19 mice also exhibited decreased NK cells and reduced tumor rejection. TBP-2 was also found to interact with various cellular target such as JAB1 and FAZF, and may be a component of a transcriptional repressor complex. See, e.g., Lee, K. N., et al., VDUP1 is required for the development of natural killer cells. Immunity 22:195-208 (2005). However, the precise mechanism of its molecular action remains to be elucidated.

Trx-2 is a 166-amino acid protein that contains a 60-amino acid residue N-terminal translocation sequence that directs it to the mitochondria. See, e.g., Spyroung, M., et al., Cloning and expression of a novel mammalian thioredoxin. J. Biol. Chem. 272: 2936-2941 (1997). Trx-2 is expressed uniquely in mitochondria, where it regulates the mitochondrial redox state and plays an important role in cell proliferation. Trx-2-deficient cells fail into apoptosis via the mitochondria-mediated apoptosis signaling pathway. See, e.g., Noon, L., et al., The absence of mitochondrial thioredoxin-2 causes massive apoptosis and early embryonic lethality in homozygous mice. Mol. Cell. Biol. 23:916-922 (2003). Trx-2 was found to form a complex with cytochrome c localized in the mitochondrial matrix, and the release of cytochrome c from the mitochondria was significantly enhanced when expression of Trx-2 was inhibited. The overexpression of Trx-2 produced resistance to oxidant-induced apoptosis in human osteosarcoma cells, indicating a critical role for the protein in protection against apoptosis in mitochondria. See, e.g., Chen, Y., et al., Overexpressed human mitochondrial thioredoxin confers resistance to oxidant-induced apoptosis in human osteosarcoma cells. J. Biol. Chem. 277:33242-33248 (2002).

As both Trx-1 and Trx-2 are known regulators of the manifestation of apoptosis under redox-sensitive capases, their actions may be coordinated. However, the functions of Trx-1 and Trx-2 do not seem to be capable of compensating for each other completely, since Trx-2 knockout mice were found be embryonically lethal. See, e.g., Noon, L., et al., The absence of mitochondrial thioredoxin-2 causes massive apoptosis and early embryonic lethality in homozygous mice. Mol. Cell. Biol. 23:916-922 (2003). Moreover, the different subcellular locations of both the thioredoxin reductase (TrxR) and thioredoxin (Trx) subtypes suggest that the cytoplasmic and mitochondrial systems may play different roles within cells. See, e.g., Powis, G. and Monofort, W. R. Properties and biological activities of thioredoxins. Ann. Rev. Pharmacol. Toxicol. 41:261-295 (2001).

### II. Biological Activities of the TrxR/Trx System

Physiological and effects modulated by thioredoxin (Trx) and related proteins Mammalian cells contain a glutathione (GSH)/glutaredoxin system and a thioredoxin (Trx)/thioredoxin reductase (TrxR) system as the two major antioxidant systems. The intracellular concentration of GSH is approximately 1-10 milliMolar (mM) in mammalian cells, whereas the normal reported intracellular concentration of Trx is approximately 0.1-2 μM. Accordingly, Trx may initially appear as a minor component as an intracellular antioxidant. However, Trx is a major enzyme supplying electrons to peroxiredoxins or methionine sulfoxide reductases, and acts as general protein disulfide reductase. Trx knock-out mice are embryonic lethal (see, e.g., Matsui, M., et al., Early embryonic lethality caused by targeted disruption of the mouse thioredoxin gene. Dev. Biol. 178:179-185 (1996)), thus illustrating that the Trx/TrxR system is playing an essential survival role in mammalian cells. This importance may be explained by Trx playing a crucial role in the interaction with specific target proteins including, but not limited to, the inhibition of apoptosis signal regulation kinase I (ASK1) activation (see, e.g., Saitoh, M., et al., Mammalian thioredoxin is a direct inhibitor of apoptosis signal-regulation kinase 1 (ASK1). EMBO J. 17:2596-2606 (1998)) and in the regulation of DNA binding activity of transcriptional factors such as AP-1, NF-κB and p53 for the transcriptional control of essential genes (see, e.g., Nakamura, H., et al., Redox regulation of cellular activation. Ann. Rev. Immunol. 15:351-369 (1997)). For example, during oxidative stress Trx-1 translocates from the cytosol into the nucleus where it augments DNA-binding activity of these aforementioned transcriptional factors. Alternately, the role of Trx in the defense against cellular oxidative stress or to supply the “building blocks” for DNA synthesis, via ribonucleotide reductase, is equally essential. Trx-1 and the 14 Kda Trx-like protein (TRP14) reactivates PTEN (a protein tyrosine phosphatase which reverses the action of phosphoinositide-3-kinase) by the reduction of the disulfide which is reversibly induced by hydrogen peroxide. See, e.g., Jeong, W., et al., Identification and characterization of TRP14, a thioredoxin-related protein of 14 Kda. J. Biol. Chem. 279:3142-3150 (2004). Exogenous Trx-1 has been shown to be capable of entering cells and attenuate intracellular reactive oxygen species (ROS) generation and cellular apoptosis. See, e.g., Kondo, N., et al., Redox-sensing release of human thioredoxin from T lymphocytes with negative feedback loops. J. Immunol. 172:442-448 (2004). Additionally, HMG-CoA reductase inhibitors (commonly utilized for the prevention of atherosclerosis) have also been shown to augment S-Nitrosylation of Trx-1 at Cys⁶⁹ and reduce oxidative stress. See, e.g., Haendeler, J., et al., Antioxidant effects of statins via S-nitrosylation and activation of thioredoxin in endothelial cells. Circulation 110:856-861 (2004).

### The Trx/TrxR System as a Cofactor in DNA Synthesis

The Trx/TrxR-coupled system plays a critical role in the generation of deoxyribonucleotides which are needed in DNA synthesis and essential for cell proliferation. Trx provides the electrons needed in the reduction of ribose by ribonucleotide reductase, an enzyme that catalyzes the conversion of nucleotide diphosphates into deoxyribonucleotides. Ribonucleotide reductase is necessary for DNA synthesis and cell proliferation. Diaziquone and doxorubicin have been shown to inhibit the Trx/TrxR system resulting in a concentration-dependent inhibition of cellular ribonucleotide reductase activity in human cancer cells. See, e.g., Mau, B., et al., Inhibition of cellular thioredoxin reductase by diaziquone and doxorubicin. Biochem. Pharmacol. 43:1621-1626 (1992). Similarly, the glutaredoxin/glutathione-coupled reaction also provides reducing equivalents for ribonucleotide reductase. For example, depletion of glutathione has been shown to inhibit DNA synthesis and induce apoptosis in a number of cancer cell lines. See, e.g., Dethlefsen, L. A., et al., Toxic effects of acute glutathione depletion by on murine mammary carcinoma cells. Radiat. Res. 114:215-224 (1988).

### The Role of the Trx/TrxR System in Cellular Apoptosis

Trx-1 was shown to prevent apoptosis (programmed cell death) when added to the culture medium of lymphoid cells or when its gene is transfected into these cells. Murine WEH17.2 lymphoid cells underwent apoptosis when exposed to the glucocorticoid dexamethasone or the topoisomerase I inhibitor etoposide and, to a lesser extent, when exposed to the kinase inhibitor staurosporine or thapsigarin, an inhibitor of intracellular calcium uptake. See, e.g., Powis, G., et al., Thioredoxin control of cell growth and death and the effects of inhibitors. Chem. Biol. Interact. 111:23-34 (1998). Trx levels in the cytoplasm and nucleus were increased following stable transfection of these cells with human Trx-1, and as a result the transfected cells showed resistance to apoptosis when exposed to dexamethasone and the other cytotoxic agents. The pattern of apoptosis inhibition with Trx-1 transfection was similar to that following transfection with the bcl-2 anti-apoptotic oncogene. In cooperation with redox factor-1, Trx-1 induces p53-dependent p-21 transactivation leading to cell-cycle arrest and DNA repair. See, e.g., Ueda, S., et al., Redox control of cell death. Antioxid. Redox Signal. 4:405-414 (2002). In addition, Trx-1 regulates the signaling for apoptosis by suppressing the activation of apoptosis signal-regulation kinase-1 (ASK-1). See, e.g., Nakamura, H., et al., Redox regulation of cellular activation. Ann. Rev. Immunol. 15:351-369 (1997).

The specific mechanism(s) by which Trx-2 imparts resistance to chemotherapy apoptosis in cancer cells has not been fully elucidated. Based on the current studies, one may postulate, however, that it appears increases in cellular reductive power allows ongoing protective and/or reparative reduction of proteins, DNA, cell membranes or carbohydrates that have been damaged or would otherwise be damaged by oxidative chemical species, thus counteracting of the induced cellular apoptosis from the chemotherapy and/or radiation therapy. The analogous glutaredoxin/glutathione system may also prevent apoptosis. In either instance, there is a lack of apoptotic sensitivity to normal treatment interventions that appears to be mediated by the increased Trx-2 and by glutaredoxin pathways. In the glutaredoxin mediated pathway, as an example, glutathione depletion with L-buthionine sulfoximine was shown to inhibit the growth of several breast and prostate cancer cell lines, and in rat R3230Ac mammary carcinoma cells, it markedly increased apoptosis. It is thought that mitochondrial swelling following depletion of glutathione may be the stimulus for apoptosis in these cells. See, e.g., Bigalow, J. E., et al., Glutathione depletion or radiation treatment alters respiration and induces apoptosis in R3230Ac mammary carcinoma. Adv. Exp. Med. Biol. 530:153-164 (2003). TX-2 has been shown to be a critical regulator of mitochondrial cytochrome c release and apoptosis. See, e.g., Tanaka, M., et al., Thioredoxin-2 (TX-2) is an essential gene in regulating mitochondrial-dependent apoptosis. EMBO J. 21:1695-1701 (2002).

### The Role of Trx in Stimulating Angiogenesis

Angiogenesis by cancer cells provides a growth and survival advantage that is localized to the primary as well as secondary (metastatic tumors). Malignant tumors are generally poorly vascular, however, with overexpression of angiogenesis factors, the tumor cells gain better nutrition and oxygenation, thereby promoting proliferation of cancer cells and growth of the tumor. Transfection of several different cell lines, including human breast cancer MCF-7, human colon cancer HT29, and murine WEHI7.2 lymphoma cells, with human Trx-1 produced significant increases in secretion of vascular endothelial growth factor (VEGF). See, e.g., Welch, S. J., et al., The redox protein thioredoxin-1 increases hypoxia-inducible factor 1α protein expression: Trx-1 overexpression results in increased vascular endothelial growth factor production and enhanced tumor angiogenesis. Cancer Res. 62:5089-5095 (2003). VEGF secretion was increased by 41%-77% under normoxic (20% oxygen) conditions and by 46%-79% under hypoxic (1% oxygen) conditions. In contrast, transfection with a redox-inactive Trx mutant (Cys→Ser) partially inhibited VEGF production. When Trx-1-transfected WEH17.2 cells were grown in SCID mice, VEGF levels were markedly increased and tumor angiogenesis (as measured by microvessel vascular density) was also increased by 2.5-fold, relative to wild-type WEH17.2 tumors. Id. Accordingly, there is evidence that the thioredoxin system can increase VEGF levels in cancer cells.

### Role of Trx in Stimulating Cell Proliferation

Exposure to Trx-1 was shown to stimulate the growth of lymphocytes, fibroblasts, and a variety of leukemic and solid tumor cell lines. See, e.g., Powis, G. and Monofort, W. R. Properties and biological activities of thioredoxins. Ann. Rev. Pharmacol. Toxicol. 41:261-295 (2001). In contrast, the previously discussed Cys→Ser redox mutant at 50-fold higher concentrations, did not stimulate cell growth. While the mechanisms for this proliferative effect are not fully elucidated, there is evidence that such Trx-mediated increases in cell proliferation are multifactorial, and are related to both the increased production of various cytokines (e.g., IL-1, IL-2, and tumor necrosis factor α (TNFα)) and the potentiation of growth factor activity (e.g., basic fibroblast growth factor (bFGF)). Additionally, there is thought to also be increased DNA synthesis and transcription, as well.

### The Antioxidant Effects of Trx

Glutathione peroxidase and membrane peroxidases play a highly important role in protecting cells against the damaging effects of reactive oxygen species (ROS) including, but not limited to, oxygen radicals and peroxides. See, e.g., Bigalow, J. E., et al., The importance of peroxide and superoxide in the x-ray response. Int. J. Radiat. Oncol. Biol. Phys. 22:665-669 (1992). These enzymes utilize use thiol groups as an electron source for scavenging reactive oxygen species (ROS), and in the process, form homo- or heterodimers with other peroxidases through the formation of disulfide bonds with conserved cysteine residues. Thioredoxin (Trx) produces antioxidant effects primarily by serving as an electron donor for thioredoxin peroxidases. Accordingly, by the reduction of oxidized peroxidases, Trx restores the enzyme to its monomeric form, which allows the enzyme to continue its oxyradical scavenging.

Trx may also increase the expression of thioredoxin peroxidase. For example, in MCF-7 human breast cancer cells stably transfected with Trx-1, mRNA for thioredoxin peroxidase was doubled relative to wild-type and empty-vector transformed cells, and Western blots showed increased protein levels as well. Moreover, Trx-1 transfected murine WEH17.2 cells were more resistant to peroxide-induced apoptosis than wild-type and empty-vector transformed cells. However, Trx-1 transfection did not protect the cells from apoptosis induced by dexamethasone or chemotherapeutic agents. See, e.g., Berggren, M. I., et al., Thioredoxin peroxidase-1 is increase in thioredoxin-1 transfected cells and results in enhanced protection against apoptosis caused by hydrogen peroxide, but not by other agents including dexamethasone, etoposide, and deoxorubin. Arch. Biochem. Biophys. 392:103-109 (2001).

### The Role of Trx in Stimulating Transcription Factor Activity

Thioredoxin (Trx) increases the DNA-binding activity of a number of transcription factors (e.g., NF-κB, AP-1, and AP-2) and nuclear receptors (e.g., glucocorticoid and estrogen receptors). See, e.g., Nishinaka, Y., et al., Redox control of cellular functions by thioredoxin: A new therapeutic direction in host defense. Arch. Immunol. Ther. Exp. 49:285-292 (2001). By way of non-limiting example, with regard to NF-κB, Trx reduces the Cys residue of the p50 subunit in the nucleus, thus allowing it to bind to DNA. See, e.g., Mau, B., et al., Inhibition of cellular thioredoxin reductase by diaziquone and doxorubicin. Biochem. Pharmacol. 43:1621-1626 (1992). In the cytoplasm, however, Trx paradoxically interferes with NF-κB by blocking dissociation of the endogenous inhibitor IκB and interfering with signaling to IκB kinases. See, e.g., Hirota, K., et al., Distinct roles of thioredoxin in the cytoplasm and in the nucleus: A two-step mechanism of redox regulation of transcription factor nf-κB. J. Biol. Chem. 274:27891-27897 (1999). The effect of Trx on some transcription factors is mediated via reduction of Ref-1, a 37 kDa protein that also possesses DNA-repair endonuclease activity. For example, Trx reduces Ref-1, which in turn reduces cysteine residues within the fos and jun subunits of AP-1 to promote DNA binding. The redox activity of Ref-1 is found in its N-terminal domain, whereas its DNA repair activity is located among C-terminal sequences.

### Trx Binding to Cellular Proteins

Reduced Trx-1, but not its oxidized form or a catalytic site Cys→Ser redox inactive mutant, binds to a variety of cellular proteins and may regulate their biological activities. See, e.g., Powis, G. and Monofort, W. R. Properties and biological activities of thioredoxins. Ann. Rev. Pharmacol. Toxicol. 41:261-295 (2001). In addition, to NK-κB and Ref-1, Trx binds to: (i) apoptosis signal-regulating kinase 1 (ASK1), (ii) various isoforms of protein kinase C (PKC), (iii) p40 phagocyte oxidase, (iv) the nuclear glucocorticoid receptor, and (v) lipocalin. ASK1, for example, is an activator of the JNK and p38 MAP kinase pathways and is required for TFNα-mediated apoptosis. See, e.g., Ichijo, H., et al., Induction of apoptosis by ask1, a mammalian map kinase that activates jnk and p38 signaling pathways. Science 275:90-94 (1997). Trx binds to a site at the N-terminal of ASK1, thus inhibiting the kinase activity and blocking ASK1-mediated apoptosis. See, e.g., Saitoh, M., et al., Mammalian thioredoxin is a direct inhibitor of apoptosis signal-regulation kinase 1 (ask1). EMBO J. 17:2596-2606 (1998). Under conditions of oxidative stress, however, reactive oxygen species are produced that oxidize the Trx, thus promoting its dissociation from ASK1 and leading to the concomitant activation of ASK 1.

### Trx/TrxR Expression in Cancer

Various extracellular roles of thioredoxin (Trx) have been examined in cancer. As previously described, Trx was originally cloned as a cytokine-like factor named ADF. Independently, Trx was also identified as an autocrine growth factor named 3B6-IL1 produced by Epstein-Barr virus-transformed B cells (see, e.g., Wakasugi, H., et al., Epstein-Barr virus-containing B-cell line produces an interleukin 1 that it uses as a growth factor. Proc. Natl. Acad. Sci. USA 84:804-808 (1987)) or as a B cell growth factor named MP6-BCGF produced by the T cell hybridoma MP6 (see, e.g., Rosen A, et al., A CD4+ T cell line-secreted factor, growth promoting for normal and leukemic B cells, identified as thioredoxin. Int. Immunol 7:625-33 (1995)). Moreover, eosinophil cytotoxicity-enhancing factor (ECEF) was found as a truncated form of Trx, comprising the N-terminal 1-80 (or 1-84) residues of Trx (Trx80) (see, e.g., Silberstein, D. S., et al., Human eosinophil cytotoxicity-enhancing factor. Eosinophil-stimulating and dithiol reductase activities of biosynthetic (recombinant) species with COOH-terminal deletions. J. Biol. Chem. 268:913-942 (1993)) and a component of “early pregnancy factor” which was an immunosuppressive factor in pregnant female serum was also identified as Trx (see, e.g., Clarke, F. M., et al., Identification of molecules involved in the “early pregnancy factor” phenomenon. J. Reprod. Fertil. 93:525-539 (1991)). These historical reports, collectively, illustrate that Trx has various important extracellular functions.

Thioredoxin (Trx) expression is increased in a variety of human malignancies including, but not limited to, lung cancer, breast cancer, colorectal cancer, cervical cancer, hepatic cancer, pancreatic cancer, and adenocarcinoma. In addition, Trx expression has also been associated with aggressive tumor growth. This increase in expression level is likely related to changes in Trx protein structure and function. For example, in pancreatic ductal carcinoma tissue, Trx levels were found to be elevated in 24 of 32 cases, as compared to normal pancreatic tissue. Glutaredoxin levels were increased in 29 of the cases. See, e.g., Nakamura, H., et al., Expression of thioredoxin and glutaredoxin, redox-regulating proteins, in pancreatic cancer. Cancer Detect. Prev. 24:53-60 (2000). Similarly, tissue samples of primary colorectal cancer or lymph node metastases had significantly higher Trx-1 levels than normal colonic mucosa or colorectal adenomatous polyps. See, e.g., Raffel, J., et al., Increased expression of thioredoxin-1 in human colorectal cancer is associated with decreased patient survival. J. Lab. Clin. Med. 142:46-51 (2003).

In two recent studies, Trx expression was associated with aggressive tumor growth and poorer prognosis. In a study of 102 primary non-small cell lung carcinomas, tumor cell Trx expression was measured by immunohistochemistry of formalin-fixed, paraffin-embedded tissue specimens. See, e.g., Kakolyris, S., et al., Thioredoxin expression is associated with lymph node status and prognosis in early operable non-small cell lung cancer. Clin. Cancer Res. 7:3087-3091 (2001). The absence of Trx expression was significantly associated with lymph node-negative status (P=0.004) and better outcomes (P<0.05) and was found to be independent of tumor stage, grade, or histology. The investigators also concluded that these results were consistent with the proposed role of Trx as a growth promoter in some human cancers, and overexpression may be indicative of a more aggressive tumor phenotype (hence the association of Trx overexpression with nodal positivity and poorer outcomes). In another study of 37 patients with colorectal cancer, Trx-1 expression tended to increase with higher Dukes stage (P=0.077) and was significantly correlated with reduced survival (P=0.004). After adjusting for Dukes stage, Trx-1 levels remained a significant prognostic factor associated with survival (P=0.012). See, e.g., Raffel, J., et al., Increased expression of thioredoxin-1 in human colorectal cancer is associated with decreased patient survival. J. Lab. Clin. Med. 142:46-51 (2003). It should be noted that GSH levels were not determined in either of the aforementioned studies.

The relationship between thioredoxin reductase (TrxR) activity and tumor growth is less clear. Tumor cells may not need to increase expression of the TrxR enzyme, although its catalytic activity may be increased functionally. For example, human colorectal tumors were found to have 2-times higher TrxR activity than normal colonic mucosa. See, e.g., Mustacich, D. and Powis, G., Thioredoxin reductase. Biochem. J. 346:1-8 (2000). TrxR has also been reported to be elevated in human primary melanoma and to show a correlation with invasiveness. See, e.g., Schallreuter, K. U., et al., Thioredoxin reductase levels are elevated in human primary melanoma cells. Int. J. Cancer 48:15-19 (1991). Further evaluations relating TrxR enzyme levels and catalytic activity with cancer stage and outcome are required needed to fully elucidate this relationship.

### The Role of Trx in Stimulating Hypoxia-Inducible Factor (HIF)

Cancer cells are able to adapt to the hypoxic conditions found in nearly all solid tumors. Hypoxia leads to activation of hypoxia-inducible factor 1 (HIF-I), which is a transcription factor involved in development of the cancer phenotype. Specifically, HIF binds to hypoxia response elements (HRE) and induces expression of a variety of genes that serve to promote: (i) angiogenesis (e.g., VEGF); (ii) metabolic adaptation (e.g., GLUT transporters, hexokinase, and other glycolytic enzymes); and (iii) cell proliferation and survival. HIF is comprised of two subunits—HIF-1α (that is induced by hypoxia) and HIF-1β (that is expressed constitutively). Trx overexpression has been shown to significantly increase HIF-1α under both normoxic and hypoxic conditions, and this was associated with increased HRE activity demonstrated in a luciferase reporter assay as well as increased expression of HRE-regulated genes. HIF may provide tumor cells with a survival advantage under hypoxic conditions by inducing hexokinase and thus allowing glycolysis to serve as the predominant energy source. For example, surgical specimens from patients with metastatic liver cancer had fewer tumor blood vessels and higher hexokinase expression than specimens from hepatocellular carcinoma patients. Hexokinase expression was correlated with HIF-1α expression in both populations, and they co-localized in tumor cells found near necrotic regions.

### The Trx/TrxR System in Cancer Drug Resistance

As previously discussed, mammalian thioredoxin reductase (TrxR) is involved in a number of important cellular processes including, but not limited to: cell proliferation, antioxidant defense, and redox signaling. Together with glutathione reductase (GR), it is also the main enzyme providing reducing equivalents to many cellular processes. GR and TrxR are flavoproteins of the same enzyme family, but only the latter is a selenoprotein. With the catalytic site containing selenocysteine, TrxR may catalyze reduction of a wide range of substrates, but it can also be easily targeted by electrophilic compounds due to the extraordinarily high reactivity of the selenocysteine moiety. In a recent studies, the inhibition of TrxR and GR by anti-cancer alkylating agents and platinum-containing compounds was compared to the inhibition of GR. See, e.g., Wang, X., et al., Thioredoxin reductase inactivation as a pivotal mechanism of ifosfamide in cancer therapy. Eur. J. Pharmacol. 579:66-75 (2008); Wang, X., et al., Cyclophosphamide as a potent inhibitor of tumor thioredoxin reductase in vivo. Toxicol. Appl. Pharmacol. 218:88-95 (2007); Witte, A-B., et al., Inhibition of thioredoxin reductase but not of glutathione reductase by the major classes of alkylating and platinum-containing anticancer compounds. Free Rad. Biol. Med. 39:696-703 (2005). These studies found that: (i) the nitrosourea, carmustine, can inhibit both GR and TrxR; (ii) the nitrogen mustards (cyclophosphamide, chlorambucil, and melphalan) and the alkyl sulfonate (busulfan) irreversibly inhibited TrxR in a concentration- and time-dependent manner, but not GR; (iii) the oxazaphosphorine, ifosfamide, inhibited TrxR; (iv) the anthracyclines (daunorubicin and doxorubicin) were not inhibitors of TrxR; (v) cisplatin, its monohydrated complex, oxaliplatin, and transplatin irreversibly inhibited TrxR, but not GR; and (vi) carboplatin could not inhibit either TrxR or GR. Other studies have shown that the irreversible inhibition of TrxR by quinones, nitrosoureas, and 13-cis-retinoic acid is markedly similar to the inhibition of TrxR by cisplatin, oxaliplatin, and transplatin. See, e.g., Arnér, E. S. J., et al., Analysis of the inhibition of mammalian thioredoxin, thioredoxin reductase, and glutaredoxin by cis-diamminedichloroplatinum (II) and its major metabolite, the glutathione-platinum complex. Free Rad. Biol. Med. 31:1170-1178 (2001).

Studies have also shown that the highly accessible selenenylsulfide/selenolthiol motif of the TrxR enzyme can be rapidly derivatized by a number of electrophilic compounds. See, e.g., Beeker, K, et al., Thioredoxin reductase as a pathophysiological factor and drug target. Eur. J. Biochem. 262:6118-6125 (2000). These compounds include, but are not limited to: (i) cisplatin and its glutathione adduct (see, e.g., Amér, E. S. J., et al., Analysis of the inhibition of mammalian thioredoxin, thioredoxin reductase; glutaredoxin by cis-diamminedichlamplatinum (II) and its major metabolite, the glutathioneplatinum complex. Free Rad. Biol. Med. 31:1170-1178 (2001)); (ii) dinitrohalobenzenes (see, e.g., Nordberg, J., et al., Mammalian thioredoxin reductase is irreversibly inhibited by dinitrohalobenzenes by alkylation of both the redox active selenocysteine and its neighboring cysteine residue. J. Biol. Chem. 273:10835-10842 (1998)); (iii) gold compounds (see, e.g., Gromer, S., et al., Human placenta thioredoxin reductase: Isolation of the selenoenzyme, steady state kinetics, inhibition by therapeutic gold compounds. J. Biol. Chem. 273:20096-20101 (1998)); (iv) organochalogenides (see, e.g., Engman, L., et al., Water-soluble organatellurium compounds inhibit thioredoxin reductase and the growth of human cancer cells. Anticancer Drug. Des. 15:323-330 (2000)); (v) different naphthazarin derivatives (see, e.g., Dessolin, I., et al., Bromination studies of the 2,3-dimethylnaphthazarin core allowing easy access to naphthazarin derivatives. J. Org. Chem. 66:5616-5619 (2001)); (vi) certain nitrosoureas (see, e.g., Sehallreuter, K. U., et al., The mechanism of action of the nitrosourea anti-tumor drugs and thioredoxin reductase, glutathione reductase and ribonucleotide reductase. Biochim. Biophys. Acta 1054:14-20 (1990)); and (vii) general thiol or selenol alkylating agents such as C-vinylpyridine, iodoacetamide or iodoacetic acid (see, e.g., Nordberg, J., et al., Mammalian thioredoxin reductase is irreversibly inhibited by dinitrohalobenzenes by alkylation of both the redox active selenocysteine and its neighboring cysteine residue. J. Biol. Chem. 273:10835-10842 (1998)).

Similarly, several lines of evidence suggest that thioredoxin (Trx) may also be necessary, but is not sufficient in toto, for conferring resistance to many chemotherapeutic drugs. This evidence includes, but is not limited to: (i) the resistance of adult T-cell leukemia cell lines to doxorubicin and ovarian cancer cell lines to cisplatin has been associated with increased intracellular Trx-1 levels; (ii) hepatocellular carcinoma cells with increased Trx-1 levels were less sensitive cisplatin (but not less sensitive to doxorubicin or mitomycin C); (iii) Trx-1 mRNA and protein levels were increased by 4- to 6-fold in bladder and prostate cancer cells made resistant to cisplatin, but lowering Trx-1 levels with an antisense plasmid restored sensitivity to cisplatin and increased sensitivity to several other cytotoxic drugs; (iv) Trx-1 levels were elevated in cisplatin-resistant gastric and colon cancer cells; and (v) stable transfection of fibrosarcoma cells with Trx-1 resulted in increased cisplatin resistance. See, e.g., Biaglow, J. E. and Miller, R. A., The thioredoxin reductase/thioredoxin system. Cancer Biol. Ther. 4:6-13 (2005).

Glutathione may also play a role in anti-cancer drug resistance. Glutathione-S-transferases catalyze the conjugation of glutathione to many electrophilic compounds, and can be upregulated by a variety of cancer drugs. Glutathione-S-transferases possess selenium-independent peroxidase activity. Mμ also has glutaredoxin activity. Some agents are substrates for glutathione-S-transferase and are directly inactivated by glutathione conjugation, thus leading to resistance. Examples of enzyme substrates include melphalan, carmustine (BCNU), and nitrogen mustard. In a panel of cancer cell lines, glutathione-S-transferase expression was correlated inversely with sensitivity to alkylating agents. Other drugs that upregulate glutathione-S-transferase may become resistant, because the enzyme also inhibits the MAP kinase pathway. These agents require a functional MAP kinase, specifically JNK and p38 activity, to induce an apoptotic response. See, e.g., Townsend, D. M. and Tew, K. D., The role of glutathione-S-transferase in anti-cancer drug resistance. Oncogene 22:7369-7375 (2003).

### Targeting Trx/TrxR-Coupled Reactions

The biological activities of Trx/TrxR and their apparent relevance to aggressive tumor growth suggest that this system may be an attractive target for cancer therapy. Either individual enzymes or substrates can be altered. In cells that do not contain glutaredoxin, depletion of hexose monophosphate shunt (HMPS)-generated NADPH or, alternately, direct interaction with Trx or TrxR may prove to be viable approaches to blocking HMPS/Trx/TrxR-coupled reactions. In cells where glutaredoxin is present, its reducing activity also may need to be targeted through depletion of glutathione.

### Thioredoxin in Plasma or Serum as an Oxidative Metabolism Biological Marker

Thioredoxin 1 (Trx-1) is released by cells in response to changes in oxidative metabolism. See, e.g., Kondo N, et al., Redox-sensing release of human thioredoxin from T lymphocytes with negative feedback loops. J. Immunol. 172:442-448 (2004). Plasma or serum levels of Trx are measurable by a sensitive sandwich enzyme-linked immunosorbent assay (ELISA). Serum plasma levels of Trx are good markers for changes in oxidative metabolism in a variety of disorders. See, e.g., Burke-Gaffney, A., et al., Thioredoxin: friend or foe in human diseases? Trends Pharmacol. Sci. 26:398-404 (2004). For example, plasma levels of Trx are elevated in patients with acquired immunodeficiency syndrome (AIDS) and negatively correlated with the intracellular levels of GSH, suggesting that the HIV-infected individuals with AIDS. See, e.g., Nakamura, H., e t al., Elevation of plasma thioredoxin levels in HIV-infected individuals. Int. Immunol. 8:603-611 (1996). In patients with type C chronic hepatitis, serum levels of Trx and ferritin are good markers for the efficacy of interferon therapy. See, e.g., Sumida, Y., et al., Serum thioredoxin levels as an indicator of oxidative stress in patients with hepatitis C virus infection. J. Hepatol. 33:616-622 (2001). In the case of cancer, serum levels of Trx are elevated in patients with hepatocellular carcinoma (see, e.g., Miyazaki, K., et al., Elevated serum levels of serum thioredoxin in patients with hepatocellular carcinoma. Biotherapy 11:277-288 (1998)) and pancreatic cancer (see, e.g., Nakmura, H., et al., Expression of thioredoxin and glutaredoxin, redox-regulating proteins, in pancreatic cancer. Cancer Detect. Prev. 24:53-40 (2000)). The serum levels of Trx decrease after the removal of the main tumor, suggesting that cancer tissues are the main source of the elevated Trx in serum. See, e.g., Miyazaki, K., et al., Elevated serum levels of serum thioredoxin in patients with hepatocellular carcinoma. Biotherapy 11:277-288 (1998).

### The Use of Trx Therapy in Cancer Patients

Since Trx shows anti-inflammatory effect in circulation, the clinical application of Trx-based therapy is now planned, especially because Trx has been shown to block neutrophil infiltration into the inflammatory site. For example, the administration of recombinant human Trx (rhTrx) inhibits bleomycin or inflammatory cytokine-induced interstitial pneumonia. See, e.g., Hoshino, T., et al., Redox-active protein thioredoxin prevents proinflammatory cytokine- or bleomycin-induced lung injury. Am. J. Respir. Crit. Care Med. 168:1075-1083 (2003). Therefore, acute respiratory distress syndrome (ARDS)/acute lung injury (ALI) is one disorder which is a good target for Trx therapy. ARDS/ALI is caused by various etiologies including anti-cancer agents such as gefitinib, a molecular-targeted agent that inhibits epidermal growth factor receptor (EGFR) tyrosine kinase. The safety of Trx therapy in cancer patients in currently being examined. Although the intracellular expression of Trx in cancer tissues is associated with, e.g., resistance to anti-cancer agents (see, e.g., Yokomizo, A., et al., Cellular levels of thioredoxin associated with drug sensitivity to cisplatin, mitomycin C, deoxrubicin, and etoposide. Cancer Res. 55:4293-4296 (1995); Sasada, T., et al., Redox control and resistance to cis-diamminedichloroplatinum (II) (CDDP); protective effect of human thioredoxin against CDDP-induced cytotoxicity. J. Clin. Investig. 97:2268-2276 (1996)), there is no evidence showing that exogenously administered rhTrx promotes the growth of cancer. For example, there is no promoting effect of administered rhTrx on the growth of the tumor planted in nude mice. In addition, administered rhTrx has no inhibitory effect on the anti-cancer agent to suppress the tumor growth in nude mice. It may be explained by that the cellular uptake of exogenous Trx is quite limited and administered Trx in plasma immediately becomes the oxidized form which has no tumor growth stimulatory activity as previously mentioned.

Thioredoxin 1 (Trx-1) expression is enhanced in cancer tissues and now inhibitors for Trx and/or thioredoxin reductase (TrxR) are studied as new anti-cancer agents. See, e.g., Powis, G., Properties and biological activities of thioredoxin. Annu. Rev. Phamacol. Toxicol. 41:261-295 (2001). From this aspect, Trx gene therapy may be dangerous in cancer-bearing patients. In contrast, the administration of rhTrx may be safe and applicable even in cancer-bearing patients to attenuate the inflammatory disorders associated with the leukocyte infiltration.

The present invention discloses compounds that are useful to mitigate or prevent deleterious chemotherapy-induced, deleterious physiological side-effects in a patient with cancer who is receiving one or more chemotherapeutic agents. The compounds possess the following generic structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

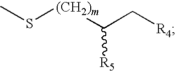

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

In another embodiment, the present invention discloses a method to mitigate or prevent chemotherapy-induced, deleterious physiological side-effects in a patient with cancer who is receiving one or more chemotherapeutic agents, with the method comprising the administration of a medically-sufficient amount of a compound having the structural formula: X—S—S—R₁—R₂.

In another embodiment, the present invention discloses pharmaceutical formulations containing one or more novel compounds possessing the structural formula: X—S—S—R₁—R₂, as the active agent, combined with one or more pharmaceutically-acceptable excipients, fillers, diluents or additives to form a pharmaceutically-acceptable formulation which is suitable for administration to human patients.

The compounds of the present invention are preferably formulated prior to administration. Therefore, another aspect of the present invention is a pharmaceutical formulation comprising a compound of Formula I and a pharmaceutically acceptable carrier, diluent, or excipient. The present pharmaceutical formulations are prepared by known procedures using well-known and readily available ingredients. In making the compositions of the present invention, the active ingredient will usually be mixed with a carrier; or dissolved or suspended in a solvent; or enclosed within a carrier, which may be in the form of a capsule, sachet, paper, or other container. The compositions can be in the form of tablets, pills, powders, lozenges, sachets, cachets, elixirs, suspensions, emulsions, solutions, syrups, aerosols, ointments containing, for example up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragacanth, gelatin, calcium silicate, microcrystalline cellulose, polyvinylpyrrolidone (PVP), dimethylacetamide (DMA), dimethylisosorbide (DMI), N-methylpyrrolidinone (NMP), cellulose, water syrup, methyl cellulose, methyl and propyl hydroxybenzoates, talc, magnesium stearate and mineral oil. The formulations can additionally include lubricating agents, wetting agents, emulsifying and suspending agents, preserving agents, sweetening agents, or flavoring agents. Compositions of the inventions may be formulated so as to provide quick, sustained, or delayed release of the active ingredient after administration to the patient by employing procedures well known in the art.

The compositions are preferably formulated in a unit dosage form, each dosage containing from about 5 mg to about 50,000 mg, more preferably about 25 to about 30,000 mg of the active ingredient. The most preferred unit dosage form contains about 10,000 mg of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier. The following formulation examples are illustrative only and are not intended to limit the scope of the invention in any way.

### Formulation 1

Hard gelatin capsules are prepared using the following ingredients:

| Ingredient | Amount | |
|---|---|---|
| Active Ingredient | 1000 | mg |
| Dried Starch | 800 | mg |
| Magnesium Stearate | 20 | mg |

### Formulation 2

A tablet is prepared using the following ingredients:

| Ingredient | Amount | |
|---|---|---|
| Active Ingredient | 1000 | mg |
| Microcrystalline Cellulose | 600 | mg |
| Silicon Dioxide, Fumed | 10 | mg |
| Stearic Acid | 10 | mg |

The components are blended and compressed to form tablets.

### Formulation 3

Tablets each containing Formula I compound as an active ingredient are made as follows:

| Ingredient | Amount | |
|---|---|---|
| Active Ingredient | 1000 | mg |
| Starch | 600 | mg |
| Microcrystalline Cellulose | 300 | mg |
| PVP | 2 | mg |
| Magnesium Stearate | 2 | mg |

The active ingredient, starch and cellulose are passed through a No. 45 mesh U.S. sieve and mixed thoroughly. The solution of PVP is mixed with the resultant powders, which are then passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed utilizing a tablet machine to yield tablets each weighing approximately 2 g.

### Formulation 4

Suspensions each containing 4,000 mg of medicament per 80 mL dose are made as follows:

| Ingredient | Amount | |
|---|---|---|
| Active Ingredient | 4,000 | mg |
| Distilled Water | 80 | mL |
| Syrup | 3 | mL |
| Benzoic Acid Solution | 1.0 | mL |
| Artificial Flavor | q.v. | |
| Artificial Color | q.v. | |
| Sodium Carboxymethyl Cellulose | 400 | mg |

The medicament is passed through a No. 45 mesh U.S. sieve and mixed with the sodium carboxymethyl cellulose and syrup to form a smooth paste. The benzoic acid solution, flavor and color are diluted with some of the water and added, with stirring. Sufficient water is then added to produce the required volume.

### Formulation 5

An intravenous formulation may be prepared as follows:

| Ingredient | Amount | |
|---|---|---|
| Active Ingredient | 10 | g |
| Purified Water | 250 | mL |
| Mannitol | 100 | mg |
| 1 N Sodium Hydroxide | 1 | mL |

Particular methods of use include administering an effective amount of the Formula I compound (or a formulation thereof) to a patient in need of treatment, or as prophylactic measures to patients in danger of exposure to one of the stated conditions. An effective amount for purposes of this application means that amount necessary to achieve the desired result. Since the Formula I compounds are of extremely low toxicity, large amounts (>40 g) can be administered safely with little or no adverse effects. Dosage may be on a single dose basis, or may be carried out on a regular schedule, depending upon the needs of the patient and the judgment of the treating physician.

In another embodiment, the compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or

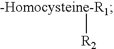

wherein R₁ and R₂ are any L- or D-amino acids; and pharmaceutically-acceptable salts and prodrugs thereof.

In still another embodiment, the compound is in the form of a pharmaceutical formulations comprising one or more compounds possessing the structural formula: X—S—S—R₁—R₂, as the active agent, combined with one or more pharmaceutically-acceptable excipients, fillers, diluents or additives to produce a formulation which is suitable for administration to human patients.

In one embodiment, the compound is a sodium salt, a potassium salt, a calcium salt, a magnesium salt, an ammonium salt, or a manganese salt.

In another embodiment, the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

In one embodiment, the chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

Another embodiment of the present invention discloses a method to modulate the intracellular balance of oxidized and reduced thioredoxin (Trx) in a patient with cancer who is receiving one or more chemotherapeutic agents, with the method comprising the administration of a medically-sufficient amount of a compound having the formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

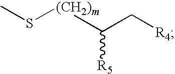

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

In another embodiment, the compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or

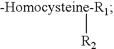

wherein R₁ and R₂ are any L- or D-amino acids; and pharmaceutically-acceptable salts and prodrugs thereof.

In another embodiment, the present invention also discloses pharmaceutical formulations containing one or more novel compounds possessing the structural formula: X—S—S—R₁—R₂, as the active agent, combined with one or more pharmaceutically-acceptable excipients, fillers, diluents or additives to form a pharmaceutically-acceptable formulation which is suitable for administration to human patients.

In yet another embodiment, the compound is a sodium salt, a potassium salt, a calcium salt, a magnesium salt, an ammonium salt, or a manganese salt.

In one embodiment, the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

In another embodiment, the chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

One embodiment of the present invention discloses a kit comprising a compound for administration and instructions for administering the compound to a patient with cancer who is receiving one or more chemotherapeutic agents, in an amount sufficient to mitigate or prevent chemotherapy-induced, physiologically-deleterious side-effects, said kit comprising the administration of a medically-sufficient amount of a compound having the structural formula:
X—S—S—R₁—R₂, wherein:
   R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

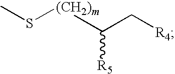

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

In another embodiment, the compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or

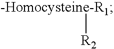

wherein R₁ and R₂ are any L- or D-amino acids; and pharmaceutically-acceptable salts and prodrugs thereof.

In still another embodiment, the compound is in the form of a pharmaceutical formulations comprising one or more compounds possessing the structural formula: X—S—S—R₁—R₂ as the active agent, combined with one or more pharmaceutically-acceptable excipients, fillers, diluents or additives to produce a formulation which is suitable for administration to human patients.

In another embodiment, the compound is a sodium salt, a potassium salt, a calcium salt, a magnesium salt, an ammonium salt, or a manganese salt.

In yet another embodiment, the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

In another embodiment, the chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

One embodiment of the present invention discloses a kit comprising a compound for administration and instructions for administering the compound to a patient with cancer who is receiving one or more chemotherapeutic agents, in an amount sufficient to modulate the intracellular balance of oxidized and reduced thioredoxin (Trx), said kit comprising the administration of a medically-sufficient amount of a compound having the structural formula:
X—S—S—R₁—R₂, wherein:
   R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or

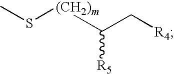

R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
   pharmaceutically-acceptable salts, prodrugs, analogs, conjugates, hydrates, solvates, polymorphs, stereoisomers (including diastereoisomers and enantiomers) and tautomers thereof.

In another embodiment, the compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or

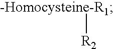

wherein R₁ and R₂ are any L- or D-amino acids; and pharmaceutically-acceptable salts and prodrugs thereof.

In one embodiment, the compound is in the form of a pharmaceutical formulations comprising one or more compounds possessing the structural formula: X—S—S—R₁—R₂ as the active agent, combined with one or more pharmaceutically-acceptable excipients, fillers, diluents or additives to produce a formulation which is suitable for administration to human patients.

In another embodiment, the compound is a sodium salt, a potassium salt, a calcium salt, a magnesium salt, an ammonium salt, or a manganese salt.

In still another embodiment, the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

In one embodiment, the chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

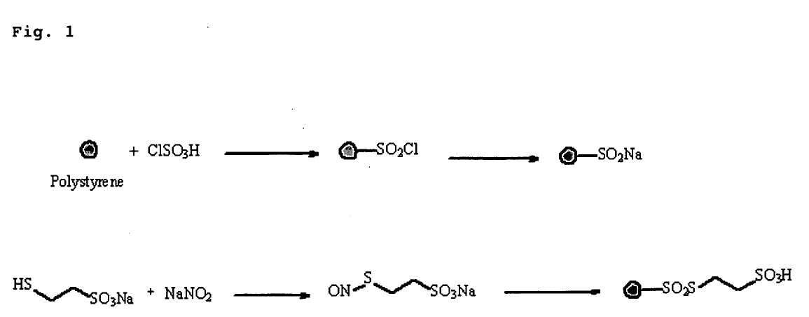

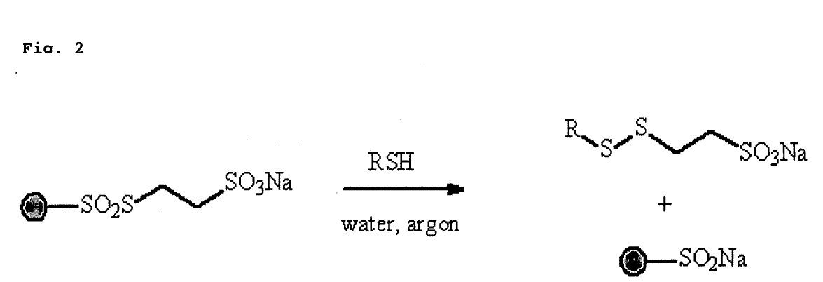

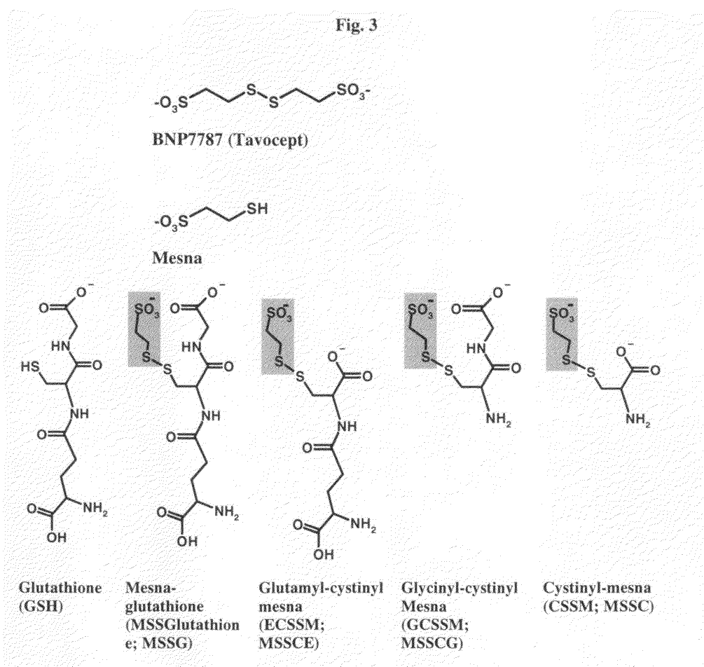

FIG. 1 illustrates a preferred synthesis of the resin-bound mesna intermediates of the present invention. The resin, preferably polystyrene microspheres of 200-400 mesh size, is functionalized with an appropriate linker, shown in Scheme 1 as sodium sulfinate. The functionalization of the resin is preferably carried out in a two-step process as shown. First, the resin is combined with a halogenated reactant to form an intermediate sulfinyl chloride linked resin, then a substitution reaction forms the sulfinate-linked resin.

2-mercaptoethane sulfonate sodium is then functionalized with an appropriate leaving group, preferably a nitric oxide moiety, and then reacted with the functionalized polystyrene to form the intermediate polymer bound mesna.

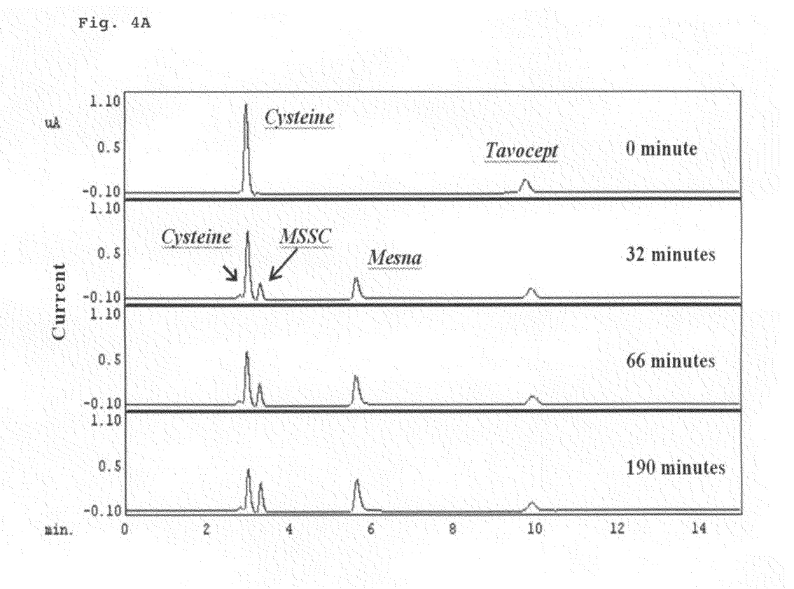

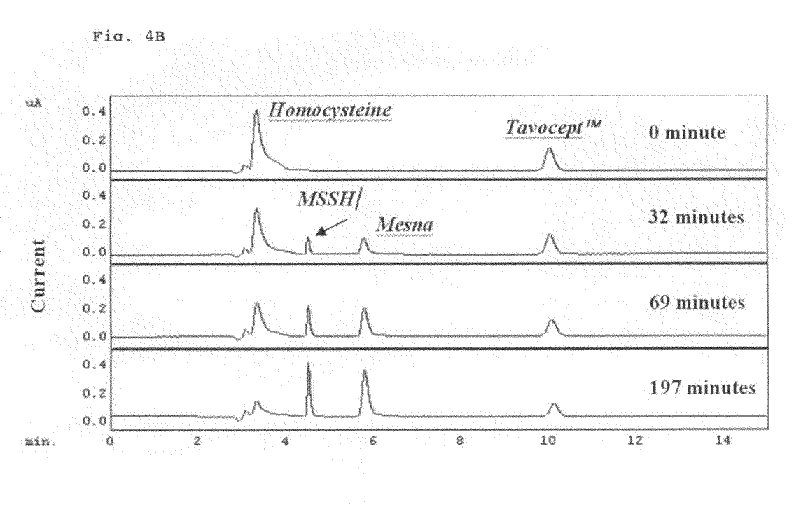

FIG. 2 illustrates the synthesis of the compounds of the present invention where R₁ is ethyl and R₂ is sulfonate. As shown, the synthetic process is a one-step, single pot process in which the polymer bound mesna is reacted with a sulfur-containing amino acid, preferably cysteine, homocysteine or glutathione; or by a short-chain peptide having 2-10 amino acids, at least one of which is a sulfur-containing amino acid. Configuration of the reactant amino acid(s) may be pure L-enantiomer, pure D-enantiomer, or a racemic mixture of the D and L stereoisomers.

After separation of the resin by conventional methods, virtually pure compound is obtained in high yields. The polymer bound sulfinate can be used again in the same or similar reactions.

Preferred compounds include those compounds where X is selected from the group consisting of: cysteine (cys); homocysteine (h-cys); glutathione (GSH); glutamic acid (glu); and short-chain peptides including cyteinyl glycine (cys-gly); glycinyl cysteine (gly-cys); glu-cys; cys-glu; glu-gly; and gly-glu. As stated above, the optical configuration of the amino acids can be the levorotatory (L) configuration, the dextrorotatory (D) configuration, or a racemic mixture thereof. Most preferred is the more active, naturally-occurring L-isomer in each case. The structures of several mesna heteroconjugates are illustrated in FIG. 3, wherein the mesna portion of the molecule is highlighted in gray. Detailed examples of the synthesis of certain mesna heteroconjugates of the present invention are set forth below.

### SPECIFIC EXAMPLES

The following Specific Examples illustrate one preferred synthesis methodology of some of the mesna heteroconjugate compounds of the present invention. These examples are disclosed for illustrative purposes only, and are not to be construed as limiting the scope of the invention in any manner.

### I. Preparation Of Resin Bound Mesna Intermediate From Sodium 2-Mercaptoethane Sulfonate

A mixture of polystyrene resin (5.0 g, Fluka, 200-400 mesh; 1% divinylbenzene) and chlorosulfonic acid (100 g) in 300 mL dichloromethane was stirred at room temperature under argon for approximately four hours, and then heated to reflux overnight. The resin was isolated by filtration while the reaction was allowed to cool to room temperature. Once the reaction temperature had cooled to room temperature, it was washed with dichloromethane (100 mL), acetonitrile (100 mL), and cold water (200 mL) sequentially. The pale brown-colored resin was then dried under high vacuum to give 9.04 g poly(styrene p-sulfonyl chloride) with 93% yield.

Poly(styrene p-sulfonyl chloride) resin (9.04 g) was suspended in 200 mL aqueous solution of sodium sulfite (60 g) and stirred at 60° C. for approximately 24 hours, isolated by filtration, washed with 200 mL water, and dried to give 8.4 gram product of mono sodium, polystyrene p-sulfinate with 99% yield.

To a solution of hydrochloric acid (2 N, 40 mL) bubbled with argon was added sodium 2-mercaptoethane sulfonate (6.56 g). The reaction solution was cooled to 0° C. in an ice bath. 20 mL aqueous solution of sodium nitrite (2.76 g) was added slowly. The reaction solution turned red and was stirred for approximately 40 minutes after the addition. The mono sodium, polystyrene p-sulfinate (3.8 g) was added and the mixture was stirred at room temperature for approximately 16 hours. The resulting polystyrene p-sulfinate bound 2-mercaptoethane sulfonic acid sodium salt was isolated by filtration, rinsed with water and dried to give 3.9 g of the title intermediate.

### II. Synthesis of L-Cysteine-Mesna Disulfide

L-Cysteine (0.50 g, 4.1 mmol) was dissolved in 50 mL de-ionized water bubbled with argon. Excessive polystyrene p-sulfinate bound 2-mercaptoethane sulfonic acid sodium salt (about 17-fold) was added. The reaction mixture was stirred under argon for approximately 4 days until all starting material of L-cysteine was consumed. The resin was removed by filtration and was recycled to prepare more disulfides. The pH of the filtrate was adjusted to neutral and lyophilized to give 0.842 g L-Cysteine-Mesna disulfide, with 72% yield.

¹H NMR (D₂O, 300 MHz) δ 3.05-3.14 (m, 3H), 3.27-3.35 (m, 3H), 3.97-4.01 (dd, 1H, J=8.1 & 4.2 Hz).

¹³C NMR (D₂O, 75 MHz) δ 31.5, 39.0, 50.4, 53.8, 174.4.

HRMS Calcd. for C₅H₁₀NO₅S₃ Na₂ (M+Na): 305.9516; Found: 305.9495.

### III. Synthesis of DL-Homocysteine-Mesna Disulfide

DL-Cysteine (0.42 g, 3.1 mmol) was dissolved in 25 mL de-ionized water bubbled with argon. Excessive polystyrene p-sulfinate bound 2-mercaptoethane sulfonic acid sodium salt (about 8.5-fold) was added. The reaction mixture was stirred under argon for approximately 4 days until all starting material of DL-Homocysteine was consumed. The resin was removed by filtration and was recycled to prepare more disulfides. The pH of filtrate was adjusted to neutral and lyophilized. The lyophilized wet cake was then recrystallized from minimum required quantity of water to give 0.293 g (32%) DL-Homocysteine-Mesna disulfide

¹H NMR (D₂O, 300 MHz) δ 2.27-2.43 (m, 2H), 2.85-2.9 (m, 2H), 3.01-3.07 (m, 2H), 3.26-3.31 (m, 2H), 4.11 (t, 1H, J=6.3 Hz).

¹³C NMR (D₂O, 75 MHz) δ 29.4, 31.7, 32.5, 50.4, 52.1, 172.4.

HRMS Calcd. for C₆H₁₄NO₅S₃ (M-Na+2H): 276.0034; Found: 276.0029.

### IV. Synthesis of Glutathione-Mesna Disulfide

Glutathione (0.54 g, 1.76 mmol) was dissolved in 25 mL de-ionized water bubbled with argon. Excessive polystyrene p-sulfinate bound 2-mercaptoethane sulfonic acid sodium salt (about 15-fold) was added. The reaction mixture was stirred under argon for 4 approximately days until all starting material of glutathione was consumed. The resin was removed by filtration and was recycled to prepare more disulfides. The pH of filtrate was adjusted to neutral and lyophilized to give 486 mg Glutathione-Mesna disulfide, with 59% yield.

¹H NMR (D₂O, 300 MHz) δ 2.07-2.14 (m, 2H), 2.47-2.54 (m, 2H), 2.94-3.08 (m, 3H), 3.25-3.32 (m, 3H), 3.66-3.71 (m, 1H), 3.75 (d, 2H, J=3.3 Hz), 4.71 (m, 1H).

¹³C NMR (D₂O, 75 MHz) δ 26.6, 31.4, 31.9 and 32.0, 38.8, 43.6, 50.6, 52.6 and 52.8, 54.3, 172.0, 174.9, 175.2, 176.5.

HRMS Calcd for C₁₂H₂₂N₃O₉S₃ (M-Na+2H): 448.0518; Found: 448.0497.

### V. Synthesis of Cysteinylglycine-Mesna Disulfide

Cysteinylglycine (226 mg, 1.27 mmol) was dissolved in 25 mL de-ionized water bubbled with argon. Excessive polystyrene p-sulfinate bound 2-mercaptoethane sulfonic acid sodium salt (about 20.5-fold) was added. The reaction mixture was stirred under argon for approximately 3 days until all starting material of cysteinyl glycine was consumed. The resin was removed by filtration and was recycled to prepare more disulfides. The pH of the filtrate was adjusted to neutral and lyophilized to give 302 mg cysteinylglycine-Mesna disulfide, with 70% yield.

¹H NMR (D₂O, 300 MHz) δ 3.07-3.19 (m, 3H), 3.27-3.39 (m, 3H), 3.93-4.1 (m, 2H), 4.41 (dd, 1H, J=8.1, 5.4 Hz).

¹³C NMR (D₂O, 75 MHz) δ 31.8, 37.9, 43.7, 50.4, 52.4, 168.5, 176.2.

### VI. Synthesis of γ-Glutamylcysteine-Mesna Disulfide

γ-Glutamylcysteine (200 mg, 0.8 mmol) was dissolved in 25 mL de-ionized water bubbled with argon. Excessive polystyrene p-sulfinate bound 2-mercaptoethane sulfonic acid sodium salt (about 32-fold) was added. The reaction mixture was stirred under argon for approximately 3 days until all starting material of γ-Glutamylcysteine was consumed. The resin was removed by filtration and was recycled to prepare more disulfides. The pH of the filtrate was adjusted to neutral and lyophilized to give 316 mg γ-Glutamylcysteine-Mesna disulfide, with 96% yield.

¹H NMR (D₂O, 300 MHz) δ 2.1-2.2 (m, 2H), 2.47-2.53 (m, 2H), 2.95-3.08 (m, 3H), 3.22-3.3 (m, 3H), 3.76 (t, J=6.3 Hz, 1H), 4.47 (dd, J=9.0 & 4.2 Hz, 1H).

¹³C NMR (D₂O, 75 MHz) δ 31.9, 39.8, 44.0, 46.4, 50.5, 54.3, 54.8, 174.6, 177.0.

### VII. Chromatography of BNP7787 and Mesna Heteroconjugates

### A. Synthesis of BNP7787, Disulfides, and Mesna Heteroconjugates

L-Cystine (CSSC), DL-homocysteine (hCys), L-homocystine (hCSShC), glutathione (GSH), glutathione disulfide (GSSG), tetrabutylammonium dihydrogenphosphate (TBAP) were purchased from Sigma. L-Cysteine (Cys) was purchased from Aldrich. HPLC grade solvents, water, and acetonitrile were obtained from Burdik & Jackson (VWR). Thioredoxin (Trx, human), glutathione, and NADPH were purchased from CalBiochem and Sigma. Thioredoxin reductase (TrxR; E.C. 1.8.1.9, bovine) was obtained from American Diagnostics. Cysteinylglycine (GCSH), cystinylglycine (GCSSCG), and γ-glutamylcysteine (ECSH) were purchased from Bachem. All other reagents were obtained from Sigma and Aldrich.

BNP7787 was prepared by a proprietary method. Mesna was purchased from Sigma. The heteroconjugates of mesna described herein were prepared by a solid-state synthesis method as outlined in Example I, infra. In brief, sodium-2-mercaptoethanesulfonate (mesna) was bound to the sulfinated polystyrene resin through a thiolsulfonic bond, then reacted with commercially available thiol-containing compounds (i.e., mesna, glutathione, cysteine, cysteinylglycine, γ-glutamylcysteine, and homocysteine) in aqueous solutions to give the high purity disulfide products: (i) glutathione-mesna disulfide (GSSM); (ii) cysteinyl-mesna disulfide (CSSM); (iii) glycinyl-cysteinyl-mesna disulfide (GCSSM); (iv) γ-glutamylcysteinyl-mesna disulfide (ECSSM); and (v) homocysteine-mesna disulfide (HSSM). The disulfide of γ-glutamylcysteine (ECSSCE) was prepared by oxygenation of an alkaline (>pH 10) aqueous solution of the thiol. The structure of ECSSCE was confirmed by proton NMR in D₂O. The final ECSSCE solution was adjusted to pH 9 before use. The specific methodologies utilized to synthesize the aforementioned mesna heteroconjugates are outlined in Examples II-VI, infra. The structural formula of the aforementioned mesna heteroconjugates are illustrated in FIG. 3, wherein the mesna portion of the molecules are highlighted in grey.

### B. Reagents and Instrumentation

Thiol disulfide exchange buffer (Buffer A) was prepared by dissolving sodium dihydrogen phosphate (100 mM) in water and adjusting the pH to 7.4 using sodium hydroxide. Trisodium citrate dihydrate (58.82 g), tetrabutylammonium dihydrogenphosphate (TBAP, 135.8 mg) and triethylamine (TEA, 0.2 mL) were added to 2 liters of water (pH adjusted to 5.0 via o-phosphoric acid (85%)); this solution was added to acetonitrile (174 mL) to provide the final HPLC mobile phase. HPLC electrochemical detection experiments were carried out on an Epsilon system from BioAnalytical Systems Inc. (West Lafayette, Ind.) with dual mercury-gold amalgam electrodes. Polishing of gold electrodes and preparation of mercury amalgam were accomplished using BAS protocols. The oxidizing electrode potential was set to 150 mV relative to Ag/AgCl reference electrode and the reducing electrode potential was optimized by correlating responses of BNP7787 peak areas with different potentials. The reducing potential of −1700 mV relative to same reference electrode for BNP7787 resulted in good sensitivity and was selected for studies herein. HPLC ultraviolet detection experiments were carried out on a Waters 600 HPLC equipped with a tunable wavelength detector (Model 486). For BNP7787 detection via HPLC UV, the wavelength was set to 254 nm. An Inertsil ODS 2 silica column (5 μM, 250×4.6 mm; G.L. Science) was used to separate the molecules of interest with a flow rate of 0.8 ml/min. Aliquots (20 μL) from the BNP7787 or Mesna reactions described below were injected manually at the indicated reaction times and the peak area was obtained by integration using Millennium (Waters) or ChromaGraph (BAS) software.

C. HPLC and Kinetic Analyses of Reactions of BNP7787 with Cysteine, Homocysteine or Glutathione

BNP7787 was incubated with, either cysteine, homocysteine or glutathione in Buffer A (pH 7.4) at 37° C. under Argon. The starting concentrations for both BNP7787 and thiol were 0.1 mM. The reactions were monitored by HPLC-EC and reaction products were identified by comparison to synthetic standards. Kinetic analyses were performed using the HPLC-UV quantification of BNP7787 that gave robust and reproducible results. The concentration of BNP7787 at the indicated reaction times was calculated based on the ratio of the peak area at the indicated time relative to the peak area at time zero. Each reaction was repeated three-times.

D. HPLC and Kinetic Analyses of Reactions of Mesna with Cystine, Homocystine or Glutathione Disulfide

Mesna was incubated with one either cystine, homocystine or glutathione disulfide in Buffer A (pH 7.4) at 37° C. under Argon. Each reaction between mesna and the various disulfides was carried out using three concentration combinations (e.g., 0.1 mM for both mesna and disulfide; 0.1 mM for mesna and 0.05 mM for the disulfide; and 0.05 mM for mesna and 0.1 mM for the disulfide). All concentration combinations were repeated three-times. Since mesna was undetectable using the HPLC-UV conditions, HPLC-EC detection was used to monitor the reactions and quantify mesna for the kinetic calculations. The concentration of mesna at the indicated reaction times was calculated based on the ratio of its peak area at the indicated time to peak area at time zero.

### E. Kinetic Equations

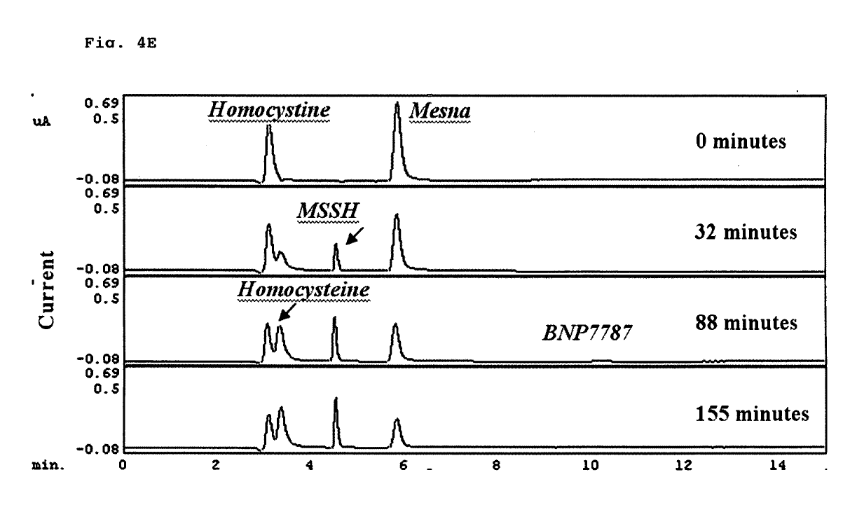

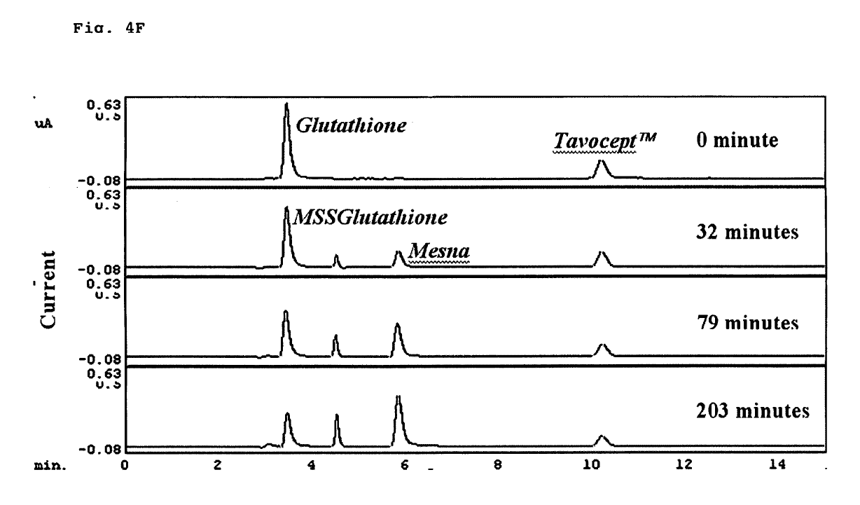

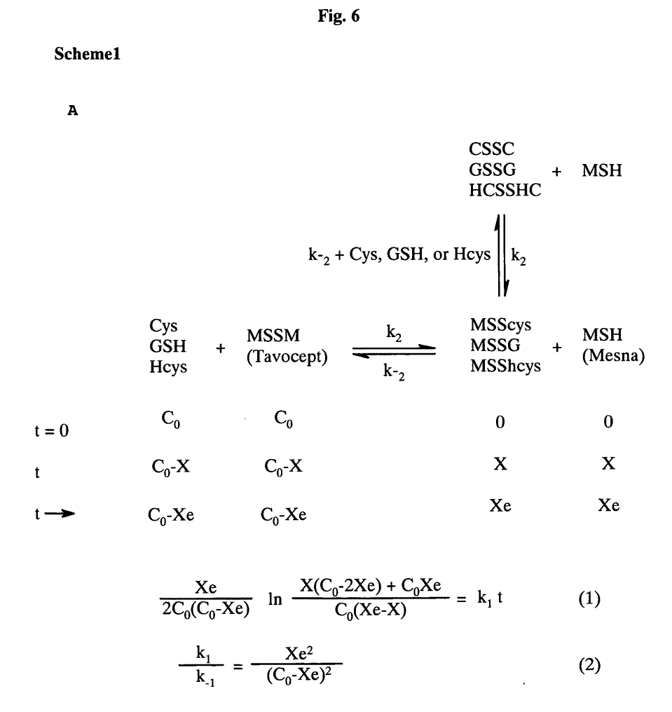

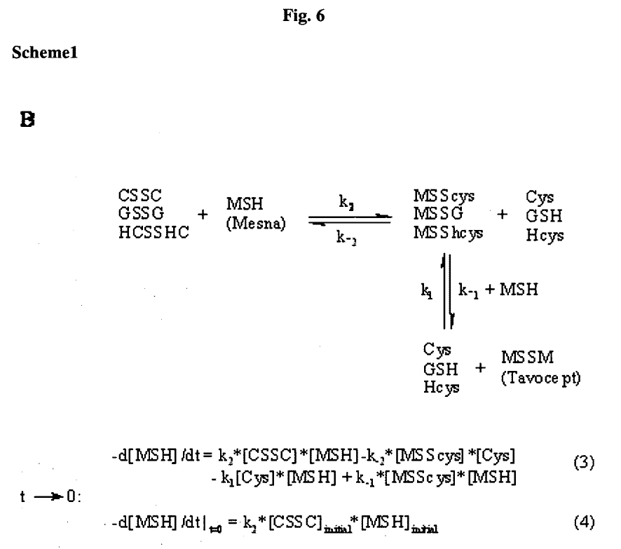

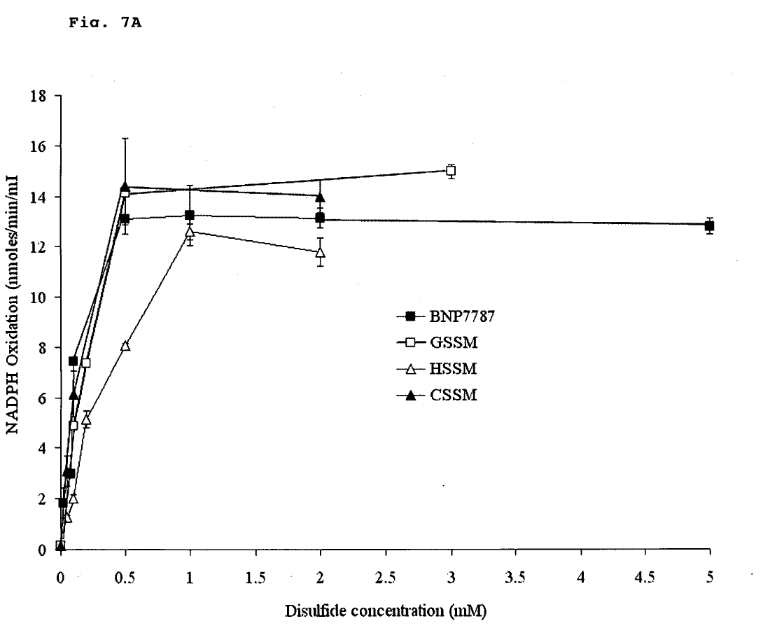

The kinetic schemes for the reactions with BNP7787 or mesna are presented in FIG. 6; Scheme 1. Once the reactions of BNP7787 with cysteine, homocysteine, or glutathione reached equilibrium, only a small amount of cystine, glutathione disulfide, and homocystine was formed; consequently, it was possible to simplify the kinetic scheme by focusing on the major reactions (i.e., the reactions characterized by rate constants k1 and k−1) and disregarding the inconsequential reactions (with rate constants, k₂ and k₋₂). Mesna reacted with cystine, homocystine, and glutathione disulfide initially forming a mixed disulfide (i.e., mesna-cysteine, mesna-homocysteine, or mesna-glutathione). The mesna heteroconjugates were formed quickly and at the later time points small amounts of BNP7787 were also detectable (see, FIG. 4D-F, later time points). The amount of BNP7787 formed was small and, therefore, the initial kinetic rate method was used to measure rate constant k₂ (see, FIG. 6, Scheme 1B, Equation 4).

### VIII. NADPH-Coupled Thioredoxin and Thioredoxin Reductase Assay

The activities of thioredoxin reductase (TrxR) and thioredoxin (Trx) was determined by monitoring NADPH oxidation at 340 nm according to the method set forth by Luthman and Holmgren (Rat liver thioredoxin and thioredoxin reductase: purification and characterization. Biochemistry 21:6628-6633 (1982)). A typical assay mixture contained TrxR Buffer (50 mM potassium phosphate, pH 7.0, 1 mM EDTA), 200 μM NADPH, 1.6 μg bovine TrxR, and one or more of the following: 4.8 μM Trx, 86 μM insulin, and one of the disulfides described herein. All disulfides were added to reactions as 10× solutions in TrxR buffer. The total volume of each reaction was 0.1 mL. Reactions were initiated by the addition of TrxR and were incubated at 25° C. for 40 min. The reactions were then analyzed using a Molecular Devices SpectraMax Plus UV/vis plate reader and the activity was calculated using a 4 minute linear portion of each assay.

### IX. Thioredoxin-Catalyzed Insulin Precipitation Assay

The effects of BNP7787 on the TrxR/Trx-catalyzed reduction of the insulin disulfide was monitored using the method of Holmgren (Thioredoxin catalyzes the reduction of insulin disulfides by dithiothreitol and dihydrolipoamide. J. Biol. Chem. 254:9627-9632 (1979)). A typical assay mixture contained TrxR Buffer (50 mM potassium phosphate, pH 7.0, 1 mM EDTA), 200 μM NADPH, 0.060 μM TrxR, 4.8 μM Trx, 330 μM dithiothreitol (DTT) and varying BNP7787 concentrations. Reactions were initiated by adding insulin (86 μM). The total volume of each reaction was 0.1 mL. Reactions were analyzed for up to 80 minutes using a Molecular Devices SpectraMax Plus UV/vis plate reader. Trx cleaves the insulin A-B chain disulfide resulting in the liberation of the insoluble free thiol form of the B chain. The TrxR/Trx reduction of the insulin disulfide requires DTT; however as many others have noted, DTT alone, does not catalyze the reduction of the insulin disulfide (data not shown).

### X. Statistical Analysis

Linear regression analyses, error calculations, and graphical representations were performed in Microsoft Excel or Kaleidograph (version 3.5). ANOVA and other statistical analyses were performed using SAS, version 8.2 and the significance level was set at 0.05.

### Specific Experimental Results

### I. Non-Enzymatic Thiol-Disulfide Exchange Reactions of BNP7787

BNP7787 reacted non-enzymatically with the physiologically relevant thiols, cysteine, homocysteine, and glutathione to form the mixed disulfides mesna-cysteine, mesna-homocysteine, mesna-glutathione, and free mesna as shown in their HPLC-EC chromatograms (see, FIG. 4A-C). These reactions are facile, occur within 30 minutes of incubation and are predictable according to the Law of Mass Action relationship. See. e.g., Hausheer, F. H., et al., New approaches to drug discovery and development: a mechanism-based approach to pharmaceutical research and its application to BNP7787, a novel chemoprotective agent. Cancer Chemother. Pharmacol. 52 (Sup. 1):S3-15 (2003). The kinetics of the reaction of BNP7787 was fastest with cysteine, followed by glutathione and then homocysteine.

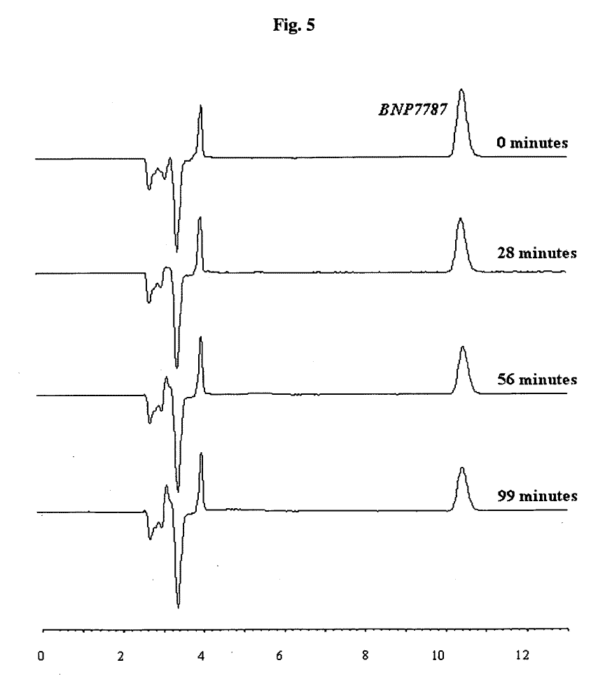

For kinetic analyses, the quantification of BNP7787 was carried out using HPLC-UV at a wavelength of 254 nm. As an example of typical HPLC-UV data, FIG. 5 illustrates chromatograms of the reaction of BNP7787 with cysteine at different incubation times. Mesna was undetectable using the HPLC-UV conditions that had been optimized for BNP7787. Cysteine and the mesna-cysteine mixed disulfide had retention times of less than 4 minutes and were either undetectable or obscured in the negative peaks from reaction buffer and, therefore, did not affect the quantification of BNP7787 which was well separated from the reaction buffer peaks. The reactions of BNP7787 with homocysteine, and with glutathione had similar HPLC-UV chromatograms as the reaction of BNP7787 with cysteine. Kinetic analyses based on quantification of BNP7787 by HPLC-UV (see, FIG. 6, Scheme 1A, Equation 2) indicated that the reaction rates of BNP7787 with the selected thiols differed. Table 2, below, provides the experimentally-derived rate constants for the reactions involving BNP7787 and the aforementioned physiologically-relevant thiols, or mesna and the aforementioned physiologically-relevant disulfides. The reaction between BNP7787 and cysteine occurred most rapidly, followed by the reaction with glutathione while the reaction with homocysteine was the slowest. The thiol-disulfide exchange reaction kinetics were affected by pH, oxygen concentration, temperature, steric/conformational properties and the relative water solubility of the reactant(s) and products. The reverse reaction with a rate constant of k₋₁, where the formed mesna reacts with the mesna-heteroconjugates to reform BNP7787, was faster than the forward reaction with a reaction rate of k₁ for all the conditions studied. In Buffer A (pH 7.4) reactions reached equilibrium in about 90 minutes.

TABLE 2

| | K₁ × 10⁶ | k₋₁ × 10⁶ | ^{a}k₂ × 10⁶ |
|---|---|---|---|
| | (μM⁻¹*S⁻¹) | (μM⁻¹*S⁻¹) | (μM⁻¹*S⁻¹) |
| BNP7787 + Cysteine | 2.17 ± 0.10 | 5.05 ± 0.23 | ^{b}negligible |
| BNP7787 + Homocysteine | 0.84 ± 0.02 | 1.73 ± 0.20 | ^{b}negligible |
| BNP7787 + Glutathione | 1.15 ± 0.03 | 1.28 ± 0.07 | ^{b}negligible |
| Mesna + Cystine | NA | NA | 9.98 ± 0.68 |
| Mesna + Homocystine | NA | NA | 2.28 ± 0.23 |
| Mesna + Glutathionedisulfide | NA | NA | 1.38 ± 0.25 |

Experiments were run in triplicate. See FIG. 3, Scheme 2 and 3 for definitions of k₁, k₋₁. and k₂
^{a}Mesna was reactive with cystine, homocystine, and glutathione disulfide initially forming a mixed disulfide (i.e., mesna-cysteine, mesna-homocysteine, or mesna-glutathione). Later mesna reacted with newly formed mixed disulfide to produce BNP7787. Therefore, the initial method has been used to measure rate constant k₂ (Equation 4 in Scheme 3).
^{b}Given that once the reactions of BNP7787 with cysteine, homocysteine, or glutathione reached equilibrium, only a small amount of cystine, glutathione disulfide, and homocystine was formed. It was possible to simplify the kinetic scheme by focusing on the major reactions (i.e., the reactions characterized by rate constants k₁ and k₋₁) and ignoring the minimal inconsequential reactions (with rate constants, k₂ and k₋₂).

### II. Non-Enzymatic Thiol-Disulfide Exchange Reactions of the BNP7787 Metabolite, Mesna

Mesna reacted with cystine, homocystine, and glutathione disulfide, to produce mesna-cysteine, mesna-homocysteine, and mesna-glutathione heteroconjugates and cysteine, homocysteine, and glutathione as shown in HPLC-EC chromatograms (FIG. 4D-F). The thiol-disulfide exchange reactions with mesna occurred within 30 minutes of incubation. The reaction of mesna with cystine was the fastest, followed by homocysteine, and then glutathione disulfide. Over time, mesna continued to react with the heteroconjugates that were formed to ultimately reform BNP7787. This continued reaction complicated the use of an equilibrium method (as used in the reactions of BNP7787 with thiols (FIG. 6; Scheme 1A; Equations 1 and 2)) to obtain the reaction rate constants for the mesna reactions with cystine, homocystine, or glutathione disulfide. Consequently, the initial rate method (mesna was not detectable using the previously described HPLC-UV method, therefore the HPLC-EC method was used) was selected to obtain rates for these reactions (FIG. 6; Scheme 1B; Equation 4). The data are consistent with the postulate that the reaction is second-order. Table 2 lists the rate constants for mesna reacting with cystine, homocystine and glutathione disulfide.

### III. BNP7787 and its Heteroconjugates as Substrates for Thioredoxin (Trx)

The activity of thioredoxin reductase (TrxR) and thioredoxin (Trx) with disulfides of mesna is described in Table 3, below. Neither BNP7787 nor any of the BNP7787-derived mesna heteroconjugates were efficient substrates for TrxR. However, all of the disulfides exhibited marked substrate behavior in the TrxR/Trx assay system. When compared, the NADPH oxidation values for Reaction A in Table 3 (i.e., TrxR alone) and the NADPH oxidation values for Reaction B in Table 3 (i.e., TrxR in combination with Trx), indicate that BNP7787 and BNP7787-derived mesna heteroconjugates are substrates for Trx. FIG. 7A depicts the concentration-dependent NADPH oxidation by TrxR, observed in the presence of Trx and selected disulfides. GSSM was a slightly more efficient substrate than BNP7787 and CSSM; whereas HSSM resulted in the least efficient NADPH oxidation. FIG. 7B illustrates that BNP7787 also inhibits Trx-catalyzed reduction of the insulin disulfide.

TABLE 3

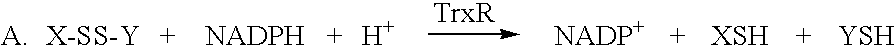

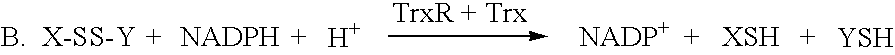

| | |
|---|---|
| | NADPH |
| Disulfide (0.5 | Oxidation (nmoles/min/mL)^{a,b} |

| mM) | Reaction A^{c} | Reaction B |
|---|---|---|
| BNP7787 | 0.3 ± 0.01 | 13.1 ± 0.2 |
| MSSGlutathione | 0.3 ± 0.02 | 14.1 ± 0.1 |
| CSSM | 0.2 ± 0.03 | 14.4 ± 0.2 |
| HSSM | 0.0 ± 0.03 | 8.6 ± 0.06 |
| ECSSM | 0.3 ± 0.02 | 9.6 ± 0.2 |
| GCSSM | 0.2 ± .04 | 15.8 ± 0.3 |

^{a}Oxidation rates were calculated from the 4 minute change in absorbance and triplicate or more assays.
^{b}A two-way ANOVA analysis was performed on the whole dataset. The difference between reaction rates for type A reactions and type B reactions is statistically significant (p-value = .0001), and affected by the disulfide substrate used in the reaction (p-value = .0001).
^{c}Zero is assigned to rates calculated from positive absorbance changes or from absorbance changes of less then .0001.

BNP7787 is a disulfide-containing compound that is expected to participate in a wide range of enzymatic and non-enzymatic thiol disulfide exchange reactions with proteins and small molecules. The Specific Experimental Results presented herein demonstrates that BNP7787 participates in facile, non-enzymatic thiol transfer reactions in vitro with thiols including, but not limited to, glutathione, cysteine and homocysteine. See, Table 2, infra. Analogously, mesna is shown to react non-enzymatically with the corresponding disulfides glutathione disulfide, cystine, and homocystine. See, Table 2, infra. These non-enzymatic reactions result in formation of BNP7787-derived mesna heteroconjugates that occur in vivo. Additionally, both BNP7787 and some BNP7787-derived mesna heteroconjugates can serve as substrates for thioredoxin, an important intracellular disulfide oxidoreductase that is responsible for helping to maintain the in vivo thiol-disulfide equilibrium.

Earlier work performed with mesna resulted in predicted metabolic conversions for this BNP7787 metabolite. See, e.g., Ormstad, K., et al., Pharmacokinetics and metabolism of sodium 2-mercaptoethanesulfonate in the rat. Cancer Res. 43:333-338 (1983) However, these earlier studies used cell lysates, rather than homogeneous enzyme preparations. In contrast, the experiments disclosed herein, utilized purified Trx system enzymes, which have now become available. Synthetically pure, BNP7787-derived mesna heteroconjugates were prepared and tested in thioredoxin assays, disclosed herein. The ECSSM and GCSSM compounds are intermediates in the synthesis and degradation of GSH, respectively. Due to the broad substrate specificity for Trx, it was anticipated that Trx would catalyze the reduction of most of the selected disulfides. It was subsequently observed that BNP7787 and all of the mesna heteroconjugates that were tested, were readily reduced by Trx in the presence of TrxR and NADPH. See, Table 3. In contrast, TrxR did not detectably reduce any of the disulfides tested. See, Table 3. It is thought that BNP7787 and the various mesna heteroconjugates (see, FIG. 3) lack the requisite structural functionalities to serve as substrates for TrxR directly, despite the fact that TrxR can accept a broad range of substrates. See, e.g., Mustacich, D. and Powis, G. Thioredoxin Reductase. Biochem. J. 346:1-8 (2000).

It was found that glutathione-mesna (GSSM), cysteine-mesna (CSSM) and glycinyl-cystinyl-mesna (GCSSM) were preferred slightly by Trx over the homocysteine-mesna (HSSM) and glutamyl-cystinyl-mesna (ECSSM) compounds; although all of the BNP7787-derived mesna heteroconjugates were found to be reasonably good substrates for Trx. The observed reduction of BNP7787 and the mesna heterodisulfides, in the presence of TrxR and Trx, was concentration dependent (see, FIG. 7A) and comparable to that observed with the standard substrate, insulin (data not shown). Additionally, it was observed that BNP7787 resulted in a dose-dependent inhibition of the Trx-catalyzed reduction of the insulin disulfide (see, FIG. 7B), indicating that the is a direct interaction between BNP7787 and Trx, in vitro. Collectively, this data suggest that Trx may be a direct participant in the metabolism of BNP7787 and BNP7787-derived mesna heteroconjugates.

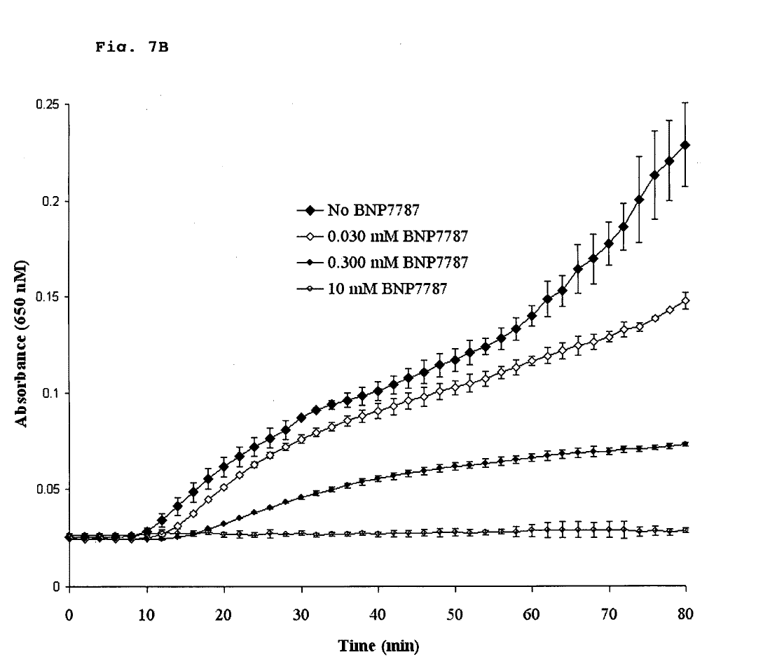

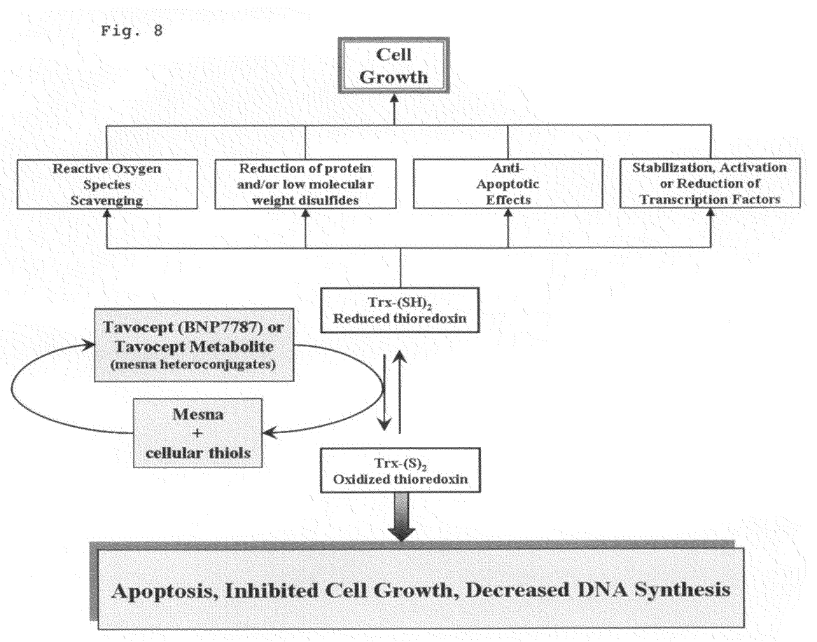

The intracellular metabolism of BNP7787 and BNP7787-derived mesna heteroconjugates may be facilitated by non-enzymatic thiol disulfide exchange reactions as well as by enzymes involved in the maintenance of cellular redox homeostasis (i.e., Trx and Grx). The identification of the Trx system in the reduction of BNP7787 and its metabolites allows future studies to be directed towards evaluation of the potential downstream effects of BNP7787 as a substrate competitor (or alternative substrate inhibitor) of Trx and as a modulator of redox maintenance by the Trx system. It is possible that, as effective substrates (or alternative substrate inhibitors) of the Trx system, that BNP7787 and the BNP7787-derived mesna heteroconjugates may modulate the intracellular balance of oxidized (i.e., inactive) and reduced (i.e., active) Trx. See, FIG. 8. A substantial increase in the inactive forms of thioredoxin could result in significant changes in the function of the natural Trx substrates that include reductases, transcription factors, and redox-dependent proteins See, FIG. 8. These effects may represent several novel mechanisms for modulating redox balance thereby affecting signal transduction, apoptosis and gene transcription.

All patents, publications, scientific articles, web sites, and the like, as well as other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicant reserves the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicant reserves the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The present invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”. The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicant reserves the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1) A method to mitigate or prevent chemotherapy-induced, deleterious physiological side-effects in a patient with cancer who is receiving one or more chemotherapeutic agents, said method comprising the administration of a medically-sufficient amount of a compound having the structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or 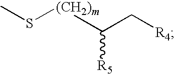
R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
pharmaceutically-acceptable salts, prodrugs, analogs, conjugates, hydrates, solvates, polymorphs, stereoisomers (including diastereoisomers and enantiomers) and tautomers thereof.

2) The method of claim 1, wherein said compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or 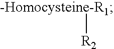
wherein R₁ and R₂ are any L- or D-amino acids; and pharmaceutically-acceptable salts and prodrugs thereof.

3) The method of claim 1 or claim 2, wherein said compound is in the form of a pharmaceutical formulations comprising one or more compounds possessing the structural formula: X—S—S—R₁—R₂ as the active agent, combined with one or more pharmaceutically-acceptable excipients, fillers, diluents or additives to produce a formulation which is suitable for administration to human patients.

4) The method of any one of claims 1-3, wherein said compound is selected from the group consisting of: a sodium salt, a potassium salt, a calcium salt, a magnesium salt, an ammonium salt, or a manganese salt.

5) The method of claim 1, wherein the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

6) The method of claim 1, wherein said chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

7) A method to modulate the intracellular balance of oxidized and reduced thioredoxin (Trx) in a patient with cancer who is receiving one or more chemotherapeutic agents, said method comprising the administration of a medically-sufficient amount of a compound having the formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or 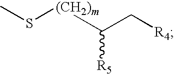
R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
pharmaceutically-acceptable salts, prodrugs, analogs, conjugates, hydrates, solvates, polymorphs, stereoisomers (including diastereoisomers and enantiomers) and tautomers thereof.

8) The method of claim 7, wherein said compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or 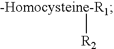
wherein R₁ and R₂ are any L- or D-amino acids; and pharmaceutically-acceptable salts and prodrugs thereof.

9) The method of claim 7 or claim 8, wherein said compound is in the form of a pharmaceutical formulations comprising one or more compounds possessing the structural formula: X—S—S—R₁—R₂ as the active agent, combined with one or more pharmaceutically-acceptable excipients, fillers, diluents or additives to produce a formulation which is suitable for administration to human patients.

10) The method of any one of claims 7-9, wherein said compound is selected from the group consisting of: a sodium salt, a potassium salt, a calcium salt, a magnesium salt, an ammonium salt, or a manganese salt.

11) The method of claim 7, wherein the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

12) The method of claim 7, wherein said chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

13) A kit comprising a compound for administration and instructions for administering said compound to a patient with cancer who is receiving one or more chemotherapeutic agents, in an amount sufficient to mitigate or prevent chemotherapy-induced, physiologically-deleterious side-effects, said kit comprising the administration of a medically-sufficient amount of a compound having the structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or 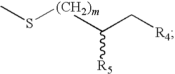
R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
pharmaceutically-acceptable salts, prodrugs, analogs, conjugates, hydrates, solvates, polymorphs, stereoisomers (including diastereoisomers and enantiomers) and tautomers thereof.

14) The kit of claim 13, wherein said compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or 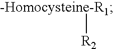
wherein R₁ and R₂ are any L- or D-amino acids; and pharmaceutically-acceptable salts and prodrugs thereof.

15) The kit of claim 13 or claim 14, wherein said compound is in the form of a pharmaceutical formulations comprising one or more compounds possessing the structural formula: X—S—S—R₁—R₂ as the active agent, combined with one or more pharmaceutically-acceptable excipients, fillers, diluents or additives to produce a formulation which is suitable for administration to human patients.

16) The kit of any one of claims 13-15, wherein said compound is selected from the group consisting of: a sodium salt, a potassium salt, a calcium salt, a magnesium salt, an ammonium salt, or a manganese salt.

17) The kit of claim 13, wherein the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma.

18) The kit of claim 13, wherein said chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors;
antivirals; and various other cytotoxic and cytostatic agents.

19) A kit comprising a compound for administration and instructions for administering said compound to a patient with cancer who is receiving one or more chemotherapeutic agents, in an amount sufficient to modulate the intracellular balance of oxidized and reduced thioredoxin (Trx), said kit comprising the administration of a medically-sufficient amount of a compound having the structural formula: X—S—S—R₁—R₂, wherein:
R₁ is a lower alkylene, wherein R₁ is optionally substituted by a member of the group consisting of: lower alkyl, aryl, hydroxy, alkoxy, aryloxy, mercapto, alkylthio or arylthio, for a corresponding hydrogen atom, or 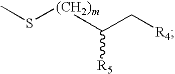
R₂ and R₄ is sulfonate or phosphonate;
R₅ is hydrogen, hydroxy, or sulfhydryl;
m is 0, 1, 2, 3, 4, 5, or 6; and
X is a sulfur-containing amino acid or a peptide consisting of from 2-10 amino acids;
or wherein X is a member of the group consisting of: lower thioalkyl (lower mercapto alkyl), lower alkylsulfonate, lower alkylphosphonate, lower alkenylsulfonate, lower alkyl, lower alkenyl, lower alkynyl, aryl, alkoxy, aryloxy, mercapto, alkylthio or hydroxy for a corresponding hydrogen atom; and
pharmaceutically-acceptable salts, prodrugs, analogs, conjugates, hydrates, solvates, polymorphs, stereoisomers (including diastereoisomers and enantiomers) and tautomers thereof.

20) The kit of claim 19, wherein said compound is a mesna heteroconjugate, comprising a 2-mercapto ethane sulfonate sodium (mesna) as a disulfide form which is conjugated with a substituent group selected from the group consisting of: -Cys, -Homocysteine, -Cys-Gly, -Cys-Glu, -Cys-Glu-Gly, -Cys-Homocysteine, -Homocysteine-Gly, -Homocysteine-Glu, -Homocysteine-Glu-Gly, or 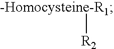
wherein R₁ and R₂ are any L- or D-amino acids; and pharmaceutically-acceptable salts and prodrugs thereof.

21) The kit of claim 19 or claim 20, wherein said compound is in the form of a pharmaceutical formulations comprising one or more compounds possessing the structural formula: X—S—S—R₁—R₂ as the active agent, combined with one or more pharmaceutically-acceptable excipients, fillers, diluents or additives to produce a formulation which is suitable for administration to human patients.

22) The kit of any one of claims 19-21, wherein said compound is selected from the group consisting of: a sodium salt, a potassium salt, a calcium salt, a magnesium salt, an ammonium salt, or a manganese salt.

23) The kit of claim 19, wherein the cancer is selected from the group consisting of: lung cancer, breast cancer, colorectal cancer, gastric cancer, esophageal cancer, ovarian cancer, cancer of the biliary tract, gallbladder cancer, cervical cancer, ovarian cancer, endometrial cancer, vaginal cancer, prostate cancer, uterine cancer, hepatic cancer, pancreatic cancer, and adenocarcinoma. cm 24) The kit of claim 19, wherein said chemotherapy agent or agents are selected from the group consisting of: fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.
